(19) United States
(12) Patent Application Publication
(10) Pub. No.: US 2022/0332792 A1
(43) Pub. Date: Oct. 20, 2022

(54) ADENO-ASSOCIATED VIRUS VECTOR PLATFORM FOR DELIVERY OF KH902 (CONBERCEPT) AND USES THEREOF

(71) Applicant: University of Massachusetts, Boston, MA (US)

(72) Inventors: Guangping Gao, Westborough, MA (US); Phillip Tai, Worcester, MA (US); Claudio Punzo, Brookline, MA (US)

(73) Assignee: University of Massachusetts, Boston, MA (US)

(21) Appl. No.: 17/639,962

(22) PCT Filed: Sep. 3, 2020
(86) PCT No.: PCT/US2020/049243

§ 371 Date: Mar. 3, 2022

Related U.S. Application Data
(60) Provisional application No. 62/895,546, filed on Sep. 4, 2019.

(51) Int. Cl. C07K 14/71; C12N 15/62; C12N 15/86; A61K 9/00

(52) U.S. Cl. CPC C07K 14/71; C12N 15/62; C12N 15/86; A61K 9/0048; C07K 2319/00; C07K 2319/30; C12N 2750/14043; C12N 2750/14071; A61K 38/00

(57) ABSTRACT

Aspects of the disclosure relate to compositions and methods for expressing one or more anti-Vascular endothelial cell growth factor (VEGF) agents in a cell or subject. In some embodiments, the disclosure provides isolated nucleic acids and rAAVs comprising a transgene encoding an anti-VEGF agent (e.g., KH902) and one or more regulatory sequences. In some embodiments, compositions described herein are useful for treating subjects having diseases associated with angiogenesis or aberrant VEGF activity/signaling.

### RELATED APPLICATION

This application is a national stage filing under 35 U.S.C. § 371 of international PCT application PCT/US2020/049243, filed Sep. 3, 2020, which claims priority under 35 U.S.C. § 119(e) to U.S. provisional patent application, U.S. Ser. No. 62/895,546, filed Sep. 4, 2019, the entire contents of each of which are incorporated herein by reference.

### BACKGROUND

KH902 is a vascular endothelial growth factor (VEGF) receptor fusion protein currently undergoing clinical trials for anti-VEGF treatment. Current challenges in anti-VEGF therapy include the need for repeated injections to sustain efficacy and long-acting formulations of anti-VEGF drugs. Therefore, there is need for development of novel methods for long- term delivery of anti-VEGF agent into targeted cells and/or tissues.

### SUMMARY

Aspects of the disclosure relate to compositions and methods for delivery of anti-VEGF agent (e.g., KH902) to cells and/or tissues (e.g., cells of a subject). The disclosure is based, in part, on isolated nucleic acids and rAAVs engineered to express a transgene encoding an anti-VEGF agent (e.g., KH902). In some embodiments, isolated nucleic acids disclosed herein include a transgene encoding an anti-vascular endothelial growth factor (e.g., an anti-VEGF) agent flanked by inverted terminal repeats (ITRs). In some embodiments, an anti-VEGF agent is a human VEGF decoy receptor. In some embodiments, the human VEGF decoy receptor comprises extracellular domain 2 of human VEGF receptor 1. In some embodiments, a human VEGF decoy receptor comprises extracellular domain 3 and 4 of human VEGF receptor 2.

In some embodiments, an anti-VEGF agent is a human VEGF receptor fusion protein. In some embodiments, a human VEGF receptor fusion protein comprises the extracellular domain 2 of human VEGF receptor 1 fused to the extracellular domain 3 and 4 of human VEGF receptor 2. In some embodiments, a human VEGF receptor fusion protein comprises a human VEGF receptor fused to an Fc portion of an immunoglobulin. In some embodiments, the human VEGF receptor fusion protein comprises the extracellular domain 2 of human VEGF receptor 1 fused to an Fc portion of an immunoglobulin. In some embodiments, wherein the human VEGF receptor fusion protein comprises the extracellular domain 3 and 4 of human VEGF receptor 2 fused to an Fc portion of an immunoglobulin. In some embodiments, the human VEGF receptor fusion protein comprises the extracellular domain 2 of human VEGF receptor 1 fused to the extracellular domain 3 and 4 of human VEGF receptor 2, and further fused to an Fc portion of an immunoglobulin. In some embodiments an anti-VEGF agent is KH902. In some embodiments, an anti-VEGF agent comprises an amino acid sequence at least 50%, at least 60%, at least 70%, at least 80%, 90%, 99% or 100% identical to amino acid sequence of SEQ ID NO: 5, or a portion thereof. In some embodiments, a transgene comprises a nucleic acid sequence at least 50%, at least 60%, at least 70%, at least 80%, 90%, 99% or 100% identical to nucleic acid sequence set forth in SEQ ID NO: 1 or a codon optimized variant thereof.

In some embodiments, an anti-VEGF agent is capable of binding to anti-vascular endothelial growth factor (VEGF) and/or placenta growth factor (PlGF).

In some embodiments, an isolated nucleic acid further comprises a promoter operably linked to a transgene. In some embodiments, the promoter comprises a cytomegalovirus (CMV) early enhancer. In some embodiments, the promoter is a chimeric cytomegalovirus (CMV)/Chicken β-actin (CB) promoter.

In some embodiments, a transgene comprises one or more introns. In some embodiments, at least one intron is positioned between a promoter and a nucleic acid sequence encoding an anti-vascular endothelial growth factor (anti-VEGF) agent. In some embodiments a transgene comprises a Kozak sequence. In some embodiments, the Kozak sequence is positioned between an intron and a transgene encoding the anti-vascular endothelial growth factor (anti-VEGF) agent.

In some embodiments, a transgene comprises a 3′ untranslated region (3′UTR). In some embodiments, the transgene further comprises one or more miRNA binding sites. In some embodiments, the one or more miRNA binding sites are positioned in a 3′UTR of the transgene. In some embodiments, at least one miRNA binding site is an immune cell-associated miRNA binding site. In some embodiments, the immune cell-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181 a, miR-223 and miR-424, miR-221, miR-222, let-7i, miR-148, and miR-152.

In some embodiments, the ITRs are adeno-associated virus ITRs of a serotype selected from the group consisting of AAV1 ITR, AAV2 ITR, AAV3 ITR, AAV4 ITR, AAV5 ITR, and AAV6 ITR.

In some embodiments, the isolated nucleic acid described herein includes a nucleic acid sequence at least 80%, 90%, 99%, or 100% identical to the nucleic acid sequence of SEQ ID NO: 2, or a portion thereof.

In some embodiments, an isolated nucleic acid as described by the disclosure is situated on a vector. In some embodiments, the vector is a plasmid, a baculoviral vector, a rAAV vector, an Anelloviral vector or a ceDNA. In some embodiments, the vector comprises a nucleic acid sequence at least 60%, 70%, 80%, 90%, 95%, 99%, or 100% identical to the nucleic acid sequence of SEQ ID NO: 3, or a portion thereof.

In some aspects, the present disclosure provides a recombinant adeno-associated virus (rAAV) vector comprising a nucleic acid comprising, in 5′ to 3′ order: (a) a 5′ AAV ITR; (b) a CMV enhancer (e.g., a CMV immediate-early enhancer) ; (c) a chicken beta-actin (CBA) promoter; (d) a chicken beta-actin intron; (e) a Kozak sequence; (f) a transgene encoding an anti-VEGF agent, wherein the anti-VEGF agent is encoded by the nucleic acid sequence in SEQ ID NO: 1; (g) a rabbit beta-globin polyA signal tail; and (h) a 3′ AAV ITR.

Also within the scope of the disclosure is a host cell comprising the isolated nucleic acid described herein. In some embodiments, a host cell is a mammalian cell, yeast cell, bacterial cell, or insect cell.

Another aspect of the disclosure relates to a recombinant adeno-associated virus (rAAV) comprising an adeno-associated virus (AAV) capsid protein and an isolated nucleic acid as described herein. In some embodiments, an rAAV comprises a capsid protein having a serotype selected from AAV1, AAV2, AAV3, AAV4, AAV5, AAV6, AAV7, AAV8, AAV9, and a variant of any of the foregoing. In some embodiments, a capsid protein has tropism for ocular tissue. In some embodiments, the ocular tissue comprises ocular neurons, retina, sclera, choroid, retina, vitreous body, macula, fovea, optic disc, lens, pupil, iris, aqueous fluid, cornea, conjunctiva ciliary body, or optic nerve.

In some embodiments, the rAAV is a single-stranded AAV (ssAAV) or a self-complementary AAV (scAAV).

Another aspect of the disclosure relates to a pharmaceutical composition comprising an isolated nucleic acid, vector, or rAAV as described herein. In some embodiments, the composition further comprises a pharmaceutically acceptable carrier. In some embodiments, the pharmaceutical composition is formulated for intravitreal injection, intravenous injection, intratumoral injection, or intramuscular injection.

In some aspects, the disclosure relates to methods of inhibiting VEGF activity in a subject in need thereof, comprising administering to the subject a therapeutically effective amount of an isolated nucleic acid, rAAV, or pharmaceutical composition as described herein.

In some aspects, the disclosure relates to a method of delivering an anti-VEGF agent in a subject in need thereof, the method comprising administering to the subject a therapeutically effective amount of an isolated nucleic acid, rAAV, or pharmaceutical composition as described herein.

In some aspects, the disclosure relates to a method of treating a neovascularization associated disease, an angiogenesis associated disease, or a VEGF-associated disease in a subject in need thereof, the method comprising administering to the subject a therapeutically effective amount of an isolated nucleic acid, rAAV, or pharmaceutical composition as described herein.

In some aspects, the disclosure provides an rAAV, or a composition comprising the rAAV for use in inhibiting VEGF activity in a subject in need thereof, wherein the rAAV comprises an adeno-associated virus (AAV) capsid protein and an isolated nucleic acid comprising a transgene encoding an anti-VEGF agent (e.g., KH902).

In some aspects, the disclosure provides an rAAV, or a composition comprising the rAAV for use in delivering an anti-VEGF agent in a subject in need thereof, wherein the rAAV comprises an adeno-associated virus (AAV) capsid protein and an isolated nucleic acid comprising a transgene encoding an anti-VEGF agent (e.g., KH902).

In some aspects, the disclosure provides an rAAV, or a composition comprising the rAAV for use in treating a neovascularization associated disease, an angiogenesis associated disease, or a VEGF-associated disease in a subject in need thereof, wherein the rAAV comprises an adeno-associated virus (AAV) capsid protein and an isolated nucleic acid comprising a transgene encoding an anti-VEGF agent (e.g., KH902).

In some aspects, the delivery of the anti-VEGF agent results in at least 10%, at least 20%, at least 30%, at least 40%, at least 50%, at least 60%, at least 70%, at least 80%, at least 90%, or 100% inhibition of VEGF activity.

In some embodiments a subject is a non-human mammal. In some embodiments, the non-human mammal is mouse, rat, cat, dog, sheep, rabbit, horse, cow, goat, pig, guinea pig, hamster, chicken, turkey, or a non-human primate. In some embodiments, the subject is a human. In some embodiments, a subject is diagnosed or is suspect of having an angiogenesis associated disease or a VEGF associated disease. In some embodiments, the disease is a tumor, a cancer, a retinopathy, a wet age-related macular degeneration (wAMD), a macular edema, a choroidal neovascularization, or a corneal neovascularization. In some embodiments, the administration is systemic administration, for example intravenous injection. In some embodiments, the administration is direct administration to ocular tissue, such as intravitreal injection, intraocular injection or topical administration.

In some embodiments, the administration results in delivery of the transgene to ocular tissue. In some embodiments, ocular tissue comprises ocular neurons, retina, sclera, choroid, retina, vitreous body, macula, fovea, optic disc, lens, pupil, iris, aqueous fluid, cornea, conjunctiva ciliary body, or optic nerve.

### BRIEF DESCRIPTION OF DRAWINGS

FIGS. 1A-1C show the rAAV-CBA-KH902 vector and sequences. The expressed rAAV vector expresses a secreted KH902 (Conbercept) and is driven by the CMV enhancer and chicken (3-actin promoter (CBA) cassette. A Kozak sequence was also designed 5′ of the start codon to enhance translation initiation. Map diagram (FIG. 1A) and read strand sequence (FIG. 1B, SEQ ID NO: 3) of the plasmid are shown. Sequences including and encompassed by the 5′-ITR and 3′-ITR are packaged into AAV virions (FIG. 1C).

FIG. 2 shows Western blot analysis of AAV-KH902-infected RPE-conditioned media. 15 μl of ARPE-19-(left) or hTERT-RPE1-(right) conditioned media for the designated conditions labeled above each lane were subjected to PAGE. Following semi-dry transfer, membranes were subjected to blotting with anti-VEGFR1 antibody (R&D Systems BAF321). 20 ng of KH902 drug (last lanes) was included as reference for each blot.

FIGS. 3A-3C show in vitro functional validation of AAV-KH902 vectors. Angiogenesis or the proliferative capacity of VEGF-stimulated (25 ng/mL) HUVECs, while in the presence of KH902; or conditioned media (diluted 1:10) of RPE cells infected with AAV2-KH902 or control GFP vector. Anti-VEGF activity was quantified by tube formation assays (FIGS. 3A and 3B) or by CCK-8 activity (FIG. 3C), respectively. *, p<0.01; **, p<0.001; ***, p<0.0001.

FIG. 4 shows that intravitreal rAAV2-KH902 injection prevents normal retinal vascular development. Neonatal mouse pups (P0-P3) were injected by intravitreal administration with rAAV2-KH902. Mice were raised in normoxic conditions (˜21% O2) and sacrificed at >P18. Retinas were mounted and stained with PECAM antibody (endothelial cells), or DAPI (DNA) and PNA (photoreceptors) and imaged from the ganglion cell side (top panels) or photoreceptor side (bottom panels).

FIGS. 5A-5C show intravitreal rAAV2-Conbercept injection prevents retinal edemas in retinopathy of prematurity. Neonatal mice (P0-P3) were injected with rAAV2-KH902 and raised for approximately 4 days in normoxic conditions (˜21% O2) and then subjected to hyperoxic conditions (75% O2) for approximately 1 week. At P12-P18, mice were brought back to normoxic conditions for 6 days and sacrificed. (FIG. 5A) Retinas were mounted and stained with anti-Isolectin B4 (vascular stain) and anti-PECAM antibodies (endothelial cells). Each treatment group (n=6) were analyzed and tabulated for the occurrence of edema (FIG. 5B) and the number of cysts (FIG. 5C).

FIGS. 6A-6B show evaluation of rAAV2-KH902 in the oxygen-induced retinopathy mouse model. FIG. 6A shows bright field images of eyes injected with rAAV2-Egfp (left column) and rAAV2-KH902:rAAV2-Egfp at a 5:1 ratio mixture (right column) and imaged immediately after dissection. Eyes in the same row are from the same animal, therefore, rAAV2-Egfp injected eyes serve as controls for the extent of pathology induction within individual animals. FIG. 6B shows fluorescence imaging of eyes from a representative mouse were then flat-mounted and stained for Isolectin-B4. Areas of positive transduction are marked by EGFP expression. rAAV2-KH902 reduces normal vascular development and aneurysm nodules; i.e., strong EGFP expression has reduced retinal vasculature. Examples of aneurysm nodules are indicated in the bottom panel (arrows).

FIGS. 7A-7B show evaluation of rAAV8-KH902 in the oxygen-induced retinopathy mouse model. FIG. 7A shows bright field images of eyes injected with rAAV8-Egfp (left column) and rAAV8-KH902:rAAV8-Egfp at a 5:1 ratio mixture (right column) and imaged immediately after dissection. Eyes in the same row are from the same animal, therefore, rAAV8-Egfp injected eyes serve as controls for the extent of pathology induction within individual animals. FIGS. 7B shows fluorescence imaging of eyes from a representative mouse were then flat-mounted and stained for Isolectin-B4. Areas of positive transduction are marked by EGFP expression. rAAV8-KH902 does not reduce normal vascular development and only modestly affects the formation of aneurysm nodules.

FIG. 8 shows percentage of rAAV treated eyes with pathologies. Mouse eyes in FIGS. 6A-6B and 7A-7B were scored for edemas or rescue. Experimental groups: rAAV2, n=10; rAAV8, n=10.

### DETAILED DESCRIPTION

In some aspects, the disclosure relates to compositions and methods for sustained long-term delivery of a vascular anti-vascular endothelial growth factor (anti-VEGF) agent (e.g., a VEGF receptor fusion protein such as KH902) to cells and/or tissue (e.g., cells and/or tissue of a subject). The disclosure is based, in part, on isolated nucleic acids (e.g., rAAV vectors) and rAAVs engineered to express transgenes encoding one or more anti-VEGF agents (e.g., a VEGF receptor fusion protein, such as KH902) or variants thereof.

### Isolated Nucleic Acids

The disclosure relates, in some aspects, to isolated nucleic acids encoding an anti-vascular endothelial growth factor (anti-VEGF) protein. Vascular endothelial growth factor (VEGF), originally known as vascular permeability factor (VPF), is a signal protein produced by cells that stimulates the formation of blood vessels. VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). VEGF's normal function is to create new blood vessels during embryonic development, new blood vessels after injury, muscle following exercise, and new vessels (collateral circulation) to bypass blocked vessels. However, aberrant VEGF activity/signaling contributes to various diseases, such as vascular diseases.

Anti-vascular endothelial growth factor therapy, also known as anti-VEGF therapy or anti-VEGF medication, is the use of medications that block vascular endothelial growth factor activity. Non-limiting examples of anti-VEGF agent include VEGF receptor fusion protein (e.g., KH902), monoclonal antibodies such as bevacizumab, antibody derivatives such as ranibizumab (Lucentis), or orally-available small molecules that inhibit the tyrosine kinases stimulated by VEGF (e.g., lapatinib, sunitinib, sorafenib, axitinib, and pazopanib).

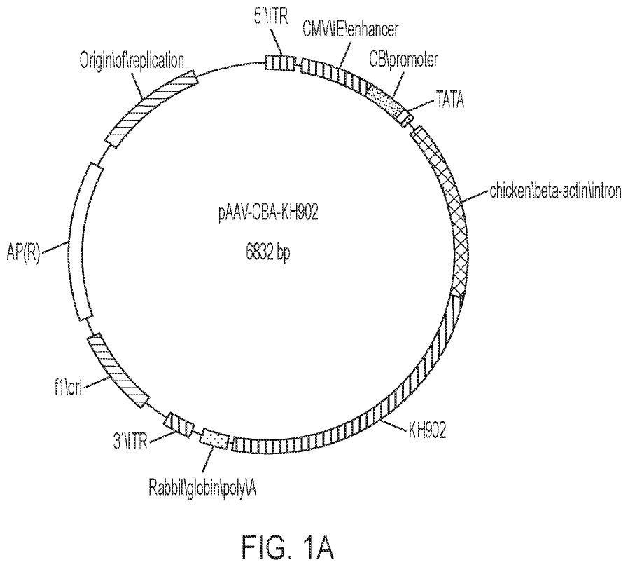

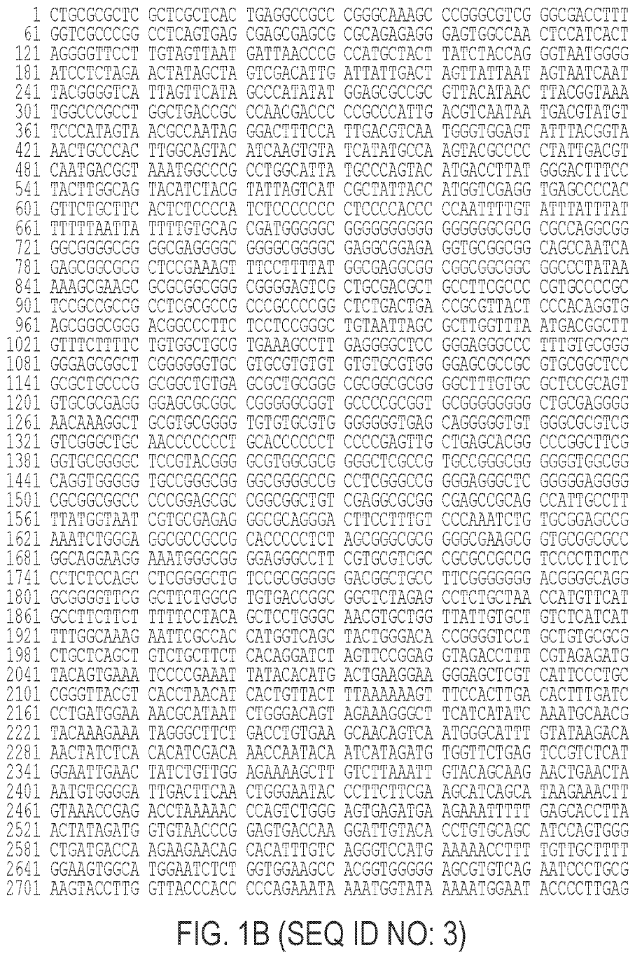

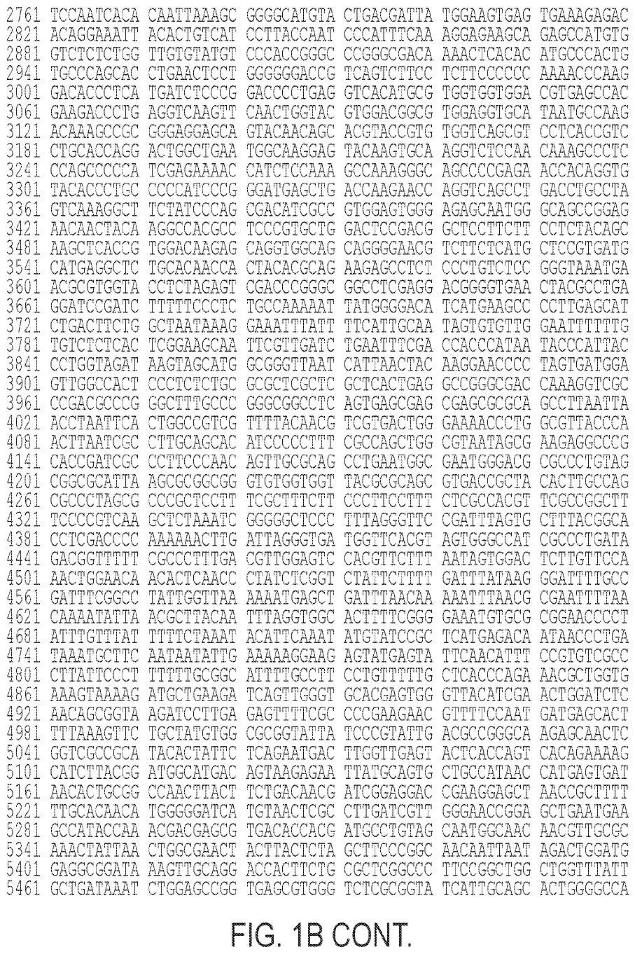

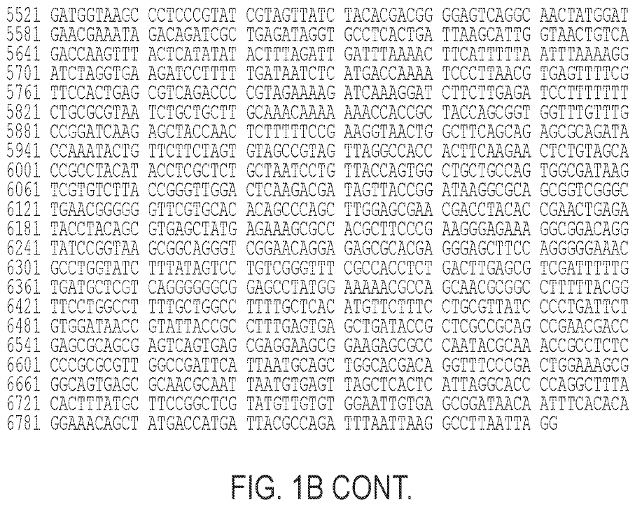

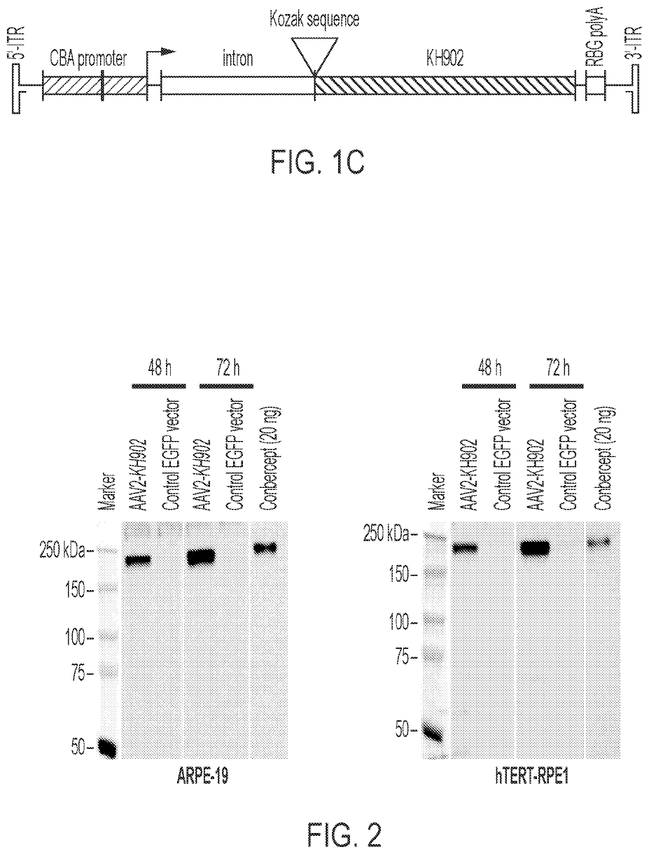

In some embodiments, isolated nucleic acids described herein comprise a transgene encoding an anti-VEGF agent. In some embodiments, the anti-VEGF agent targets (e.g., specifically binds to) a human VEGF receptor. VEGF receptors are receptors for vascular endothelial growth factor (VEGF). There are three main subtypes of VEGF receptor, numbered 1, 2 and 3. VEGFR-1, VEGFR-2, and VEGFR-3 belong to the receptor tyrosine kinase family (FIG. 1A). VEGFR-1 and -2 are primarily involved in angiogenesis, whereas VEGFR-3 are involved in hematopoiesis and lymphangiogenesis. The VEGFRs contain an approximately 750-amino-acid-residue extracellular domain, which is organized into seven immunoglobulin-like folds. Adjacent to the extracellular domain is a single transmembrane region, followed by a juxtamembrane domain, a split tyrosine-kinase domain that is interrupted by a 70-amino-acid kinase insert, and a C-terminal tail. VEGF receptor activation requires dimerization. Guided by the binding properties of the ligands, VEGFRs form both homodimers and heterodimers. Dimerization of VEGFR is accompanied by activation of receptor kinase activity, leading to autophosphorylation. Signal transduction is propagated when activated VEGF receptors phosphorylate SH2 domain-containing protein substrates. Vascular endothelial growth factor (VEGF) is an important signaling protein involved in many biological pathways (e.g., vasculogenesis and angiogenesis). The VEGF receptors have an extracellular portion consisting of 7 immunoglobulin-like domains (e.g., extracellular domain 1-7), a single transmembrane spanning region and an intracellular portion containing a split tyrosine-kinase domain. In some embodiments, human VEGF receptor 1 comprises an amino acid sequence as set forth in NCBI Accession No. NP_001153392.1, NCBI Accession No. NP_001153502.1, NCBI Accession No. NP_001153503.1, or NCBI Accession No. NP_002010.2. In some embodiments, human VEGF receptor 2 comprises an amino acid sequence as set forth in NCBI Accession No. NP_002244.1. In some embodiments, human VEGF receptor 3 comprises an amino acid sequence as set forth in NCBI Accession No. NP_002011.2, NCBI Accession No. NP_001341918.1. or NCBI Accession No. NP_891555.2. In some embodiments, an anti-VEGF agent targets (e.g., specifically binds to) a placental-derived growth factor (P1GF).

In some embodiments, the anti-VEGF agent is a human VEGF decoy receptor, or a portion thereof. A “decoy receptor” refers to a receptor that is able to recognize and bind a ligand (e.g., VEGF), but is not structurally able to signal or activate the cognate receptor complex of the ligand. The VEGF decoy receptor acts as an inhibitor, binding a ligand and keeping it from binding to its regular receptor. In some embodiments, the VEGF decoy receptor comprises one or more extracellular domains of the VEGF receptor 1 and/or VEGF receptor 2. In some embodiments, the anti-VEGF agent is a human VEGF decoy receptor fusion protein. In some embodiments, the human VEGF decoy receptor fusion protein comprises more than one extra cellular domains selected from VEGF receptor 1 and/or VEGF receptor 2 fused together. In some embodiments, the human VEGF decoy receptor fusion protein comprises a first portion including a VEGF receptor 1 fused to a VEGF receptor 2, which is further fused to second portion comprising a different protein (e.g., Fc portion of an immunoglobulin). VEGF decoy receptors and VEGF decoy receptor fusion proteins have been previously described, see. e.g., WO2007112675, and EP1767546B1, the entire contents of which are incorporated herein by reference.

In some embodiments, the human VEGF decoy receptor comprises an extracellular domain of a protein that binds VEGF. In some embodiments, the human VEGF decoy receptor comprises an extracellular domain of human VEGF receptor 1. In some embodiments, the human VEGF decoy receptor comprises extracellular domain 2 of human VEGF receptor 1. In some embodiments, the human VEGF decoy receptor comprises an extracellular domain of human VEGF receptor 2. In some embodiments, the human VEGF decoy receptor comprises extracellular domains 3 and 4 of human VEGF receptor 2.

In some embodiments, the human VEGF decoy receptor is a human VEGF receptor fusion protein. In some embodiments, the VEGF receptor fusion protein comprises an extracellular domain selected from VEGF receptor 1 or VEGF receptor 2, and one or more second extracellular domain selected from VEGF receptor 1 or VEGF receptor 2. In some embodiments, the VEGF receptor fusion protein comprises extracellular domain 2 of VEGF receptor 1, and extracellular domain 3 of VEGF receptor 2. In some embodiments, the VEGF receptor fusion protein comprises extracellular domain 2 of VEGF receptor 1, and extracellular domains 3 and 4 of VEGF receptor 2. In some embodiments, the VEGF receptor fusion protein comprises extracellular domain 2 of VEGF receptor 1, fused to extracellular domain 3 of VEGF receptor 2, and further fused to extracellular domain 4 of VEGF receptor 1. In some embodiments, the VEGF receptor fusion protein comprises extracellular domain 1 of VEGF receptor 2, fused to extracellular domain 2 of VEGF receptor 1, and further fused to extracellular domain 3 of VEGF receptor 2. In some embodiments, the VEGF receptor fusion protein comprises extracellular domain 2 of VEGF receptor 1, fused to extracellular domain 3 of VEGF receptor 2, and further fused to extracellular domain 4 of VEGF receptor 2, and further fused to extracellular domain 5 of VEGF receptor 2. In some embodiments, the VEGF receptor fusion protein comprises extracellular domain 2 of VEGF receptor 1, fused to extracellular domain 3 of VEGF receptor 2, and further fused to extracellular domain 4 of VEGF receptor 2, and further fused to extracellular domain 5 of VEGF receptor 1. In some embodiments, the fused extracellular domains of a VEGF decoy receptor are connected to one another by a linker. In some embodiments, the fused extracellular domains of a VEGF decoy receptor are connected to one another directly.

In addition, any of the VEGF receptor fusion proteins described herein may be fused to another protein. In some embodiments, the VEGF receptor fusion protein comprises a portion that is VEGF receptor (e.g., any of the VEGF decoy receptor or VEGF decoy receptor fusion protein described herein) fused to another protein to provide dimerization or multimerization properties. Non-limiting examples of the protein to provide dimerization or multimerization properties for the fusion protein is the Fc portion of an immunoglobulin. In some embodiments, the VEGF receptor fusion protein comprises a portion that is VEGF receptor (e.g., any of the VEGF decoy receptor or VEGF decoy receptor fusion protein described herein) is fused to an Fc portion of an immunoglobulin. In some embodiments, the VEGF receptor fusion protein (e.g., a VEGF decoy receptor or a VEGF decoy receptor fusion protein described herein) is fused to the other portion (e.g., an Fc domain) directly. In some embodiments, VEGF receptor fusion protein (e.g., a VEGF receptor decoy) is fused to the other portion via a linker.

Suitable linkers are known in the art. (See, e.g., Chen et al., Fusion protein linkers: property, design and functionality, Adv Drug Deliv Rev. 2013 October; 65(10):1357-69). In some embodiments, the VEGF receptor fusion protein is further fused to an Fc portion of an immunoglobulin. In some embodiments, the VEGF receptor fusion protein is KH902. KH902, also known as Conbercept (e.g., US20100272719A1, the entire contents which are incorporated herein by reference) is a decoy receptor protein constructed by fusing vascular endothelial growth factor (VEGF) receptor 1 and VEGF receptor 2 extracellular domains with the Fc region of human immunoglobulin. The size of KH902 is about 142 kD. Conbercept-mediated blockage of VEGF and placental growth factor (PIGF), which can induce neovascularization, has been proven to effectively treat wet age-related macular degeneration (wAMD) in clinical trials, including phase 3 trials, see. e.g., Liu et al., AJO, Aug. 17, 2019, the entire contents of which are incorporated herein by reference.

In some embodiments, the anti-VEGF agent comprises an amino acid sequence at least at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence as set forth in SEQ ID NO: 5. An exemplary amino acid sequence for KH902 is set forth in SEQ ID NO: 5.

(SEQ ID NO: 5)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
IPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKATP
PVLDSDGSFFLYSKLTVDKSRWQQGNVFSCSVMHEALHNHYTQKSLSLSP
GK

In some embodiments, the anti-VEGF agent comprises a portion of SEQ ID NO: 5. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 2 of VEGF receptor 1 as set forth in SEQ ID NO: 6. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 3 and 4 of VEGF receptor 2 as set forth in SEQ ID NO: 7. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 2 of VEGF receptor 1 fused to extracellular domain 2 of VEGF receptor 1 as set forth in SEQ ID NO: 8. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 2 of VEGF receptor 1 fused to an Fc portion of an immunoglobulin as set forth in SEQ ID NO:9. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 3 and 4 of VEGF receptor 2 fused to an Fc portion of an immunoglobulin as set forth in SEQ ID NO: 10. An exemplary amino acid sequence of extracellular domain 2 of VEGF receptor 1 is set forth in SEQ ID NO: 6:

(SEQ ID NO: 6)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDV

An exemplary amino acid sequence of extracellular domain 3 and 4 of VEGF receptor 2 is set forth in SEQ ID NO: 7:

(SEQ ID NO: 7)
VLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHE
KPFVAFGSGMESLVEATVGERVRIPAKYLGYPPPEIKWYKNGIPLESNHT
IKAGHVLTIMEVSERDTGNYTVILTNPISKEKQSHVVSLVVYVPP

An exemplary amino acid sequence of extracellular domain 2 of VEGF receptor 1 fused to extracellular domain 3 and 4 of VEGF receptor 2 is set forth in SEQ ID NO: 8:

(SEQ ID NO: 8)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
IPAKYLGYPPPEIKWYKNGIPLESNHTIKAGHVLTIMEVSERDTGNYTVI
LTNPISKEKQSHVVSLVVYVPP

An exemplary amino acid sequence of extracellular domain 2 of VEGF receptor 1 fused to Fc portion is set forth in SEQ ID NO: 9

(SEQ ID NO: 9)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVGPGDKTHTCPLCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKATPPVLDSDGSFFLYSKLTVDKSRWQQGNVFSCSVMHEALHNHYTQKS
LSLSPGK

An exemplary amino acid sequence of extracellular domain 3 and 4 of VEGF receptor 2 fused to Fc portion is set forth in SEQ ID NO: 10:

(SEQ ID NO: 10)
VLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHE
KPFVAFGSGMESLVEATVGERVRIPAKYLGYPPPEIKWYKNGIPLESNHT
IKAGHVLTIMEVSERDTGNYTVILTNPISKEKQSHVVSLVVYVPPGPGDK
THTCPLCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGF
YPSDIAVEWESNGQPENNYKATPPVLDSDGSFFLYSKLTVDKSRWQQGNV
FSCSVMHEALHNHYTQKSLSLSPGK

In some embodiments, the isolated nucleic acid comprises a nucleic acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 1. An exemplary coding sequence for KH902 is set forth in SEQ ID NO: 1.

(SEQ ID NO: 1)
ATGGTCAGCTACTGGGACACCGGGGTCCTGCTGTGCGCGCTGCTCAGCTG
TCTGCTTCTCACAGGATCTAGTTCCGGAGGTAGACCTTTCGTAGAGATGT
ACAGTGAAATCCCCGAAATTATACACATGACTGAAGGAAGGGAGCTCGTC
ATTCCCTGCCGGGTTACGTCACCTAACATCACTGTTACTTTAAAAAAGTT
TCCACTTGACACTTTGATCCCTGATGGAAAACGCATAATCTGGGACAGTA
GAAAGGGCTTCATCATATCAAATGCAACGTACAAAGAAATAGGGCTTCTG
ACCTGTGAAGCAACAGTCAATGGGCATTTGTATAAGACAAACTATCTCAC
ACATCGACAAACCAATACAATCATAGATGTGGTTCTGAGTCCGTCTCATG
GAATTGAACTATCTGTTGGAGAAAAGCTTGTCTTAAATTGTACAGCAAGA
ACTGAACTAAATGTGGGGATTGACTTCAACTGGGAATACCCTTCTTCGAA
GCATCAGCATAAGAAACTTGTAAACCGAGACCTAAAAACCCAGTCTGGGA
GTGAGATGAAGAAATTTTTGAGCACCTTAACTATAGATGGTGTAACCCGG
AGTGACCAAGGATTGTACACCTGTGCAGCATCCAGTGGGCTGATGACCAA
GAAGAACAGCACATTTGTCAGGGTCCATGAAAAACCTTTTGTTGCTTTTG
GAAGTGGCATGGAATCTCTGGTGGAAGCCACGGTGGGGGAGCGTGTCAGA
ATCCCTGCGAAGTACCTTGGTTACCCACCCCCAGAAATAAAATGGTATAA
AAATGGAATACCCCTTGAGTCCAATCACACAATTAAAGCGGGGCATGTAC
TGACGATTATGGAAGTGAGTGAAAGAGACACAGGAAATTACACTGTCATC
CTTACCAATCCCATTTCAAAGGAGAAGCAGAGCCATGTGGTCTCTCTGGT
TGTGTATGTCCCACCGGGCCCGGGCGACAAAACTCACACATGCCCACTGT
GCCCAGCACCTGAACTCCTGGGGGGACCGTCAGTCTTCCTCTTCCCCCCA
AAACCCAAGGACACCCTCATGATCTCCCGGACCCCTGAGGTCACATGCGT
GGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAG
TACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAA
CCACAGGTGTACACCCTGCCCCCATCCCGGGATGAGCTGACCAAGAACCA
GGTCAGCCTGACCTGCCTAGTCAAAGGCTTCTATCCCAGCGACATCGCCG
TGGAGTGGGAGAGCAATGGGCAGCCGGAGAACAACTACAAGGCCACGCCT
CCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCG
GGTAAATGA

Any of the anti-VEGF agent described herein and or a combination thereof can be expressed by an isolated nucleic acid herein. In some embodiments, the isolated nucleic acid comprises a first region encoding the extracellular domain 2 of VEGF receptor 1 and a second region encoding the extracellular domain 3 and 4 of VEGF receptor 2. In some embodiments, the isolated nucleic acid comprises a first region encoding the extracellular domain 2 of VEGF receptor 1 fused to an Fc portion of an immunoglobulin and a second region encoding the extracellular domain 3 and 4 of VEGF receptor 2 fused to an Fc portion of an immunoglobulin. In some embodiments, the first region may be positioned at any suitable location. The first region maybe positioned upstream of the second region. For example, the first region may be positioned between the first codon of the second region and 2000 nucleotides upstream of the first codon. The first region may be positioned between the first codon of the second region and 1000 nucleotides upstream of the first codon. The first region may be positioned between the first codon of the second region and 500 nucleotides upstream of the first codon. The first region may be positioned between the first codon of the second region and 250 nucleotides upstream of the first codon. The first region may be positioned between the first codon of the second region and 150 nucleotides upstream of the first codon. In other embodiments, the first region may be positioned downstream of the second region. The first region may be between the last codon of the second region and a position 2000 nucleotides downstream of the last codon.

The first region may be between the last codon of the second region and a position 1000 nucleotides downstream of the last codon. The first region may be between the last codon of second region and a position 500 nucleotides downstream of the last codon. The first region may be between the last codon of the second region and a position 250 nucleotides downstream of the last codon. The first region may be between the last codon of the second region and a position 150 nucleotides downstream of the last codon.

In some embodiments, the nucleic acid may also comprise a third region. In some embodiments, the isolated nucleic acid comprises a first region encoding the extracellular domain 2 of VEGF receptor 1, a second region encoding the extracellular domain 3 and 4 of VEGF receptor 2 and a third region encoding the extracellular domain 2 of VEGF receptor 1 fused to the extracellular domain 3 and 4 of VEGF receptor 2. In some embodiments, the isolated nucleic acid comprises a first region encoding the extracellular domain 2 of VEGF receptor 1 fused to an Fc portion of an immunoglobulin, a second region encoding the extracellular domain 3 and 4 of VEGF receptor 2 fused to an Fc portion of an immunoglobulin and a third region encoding the extracellular domain 2 of VEGF receptor 1 fused to the extracellular domain 3 and 4 of VEGF receptor 2, and further fused to an Fc portion of an immunoglobulin. In some embodiments, the third region of positioned upstream of the first codon of the first region. In some embodiments, the third region is positioned between the last codon of the first region and the first codon of the second region. In some embodiments, the third region is positioned downstream of the last codon of the second region.

In some embodiments, the various regions of an isolated nucleic acid disclosed herein are expression cassettes for expressing the anti-VEGF agent or a combination of anti-VEGF agents described herein. In some embodiments, a multicistronic expression construct comprises two or more expression cassettes encoding one or more anti-VEGF agents, or a combination of anti-VEGF agents described herein.

In some embodiments, multicistronic expression constructs are comprise expression cassettes that are positioned in different ways. For example, in some embodiments, a multicistronic expression construct is provided in which a first expression cassette (e.g., an expression cassette encoding a first anti-VEGF agent, or portion thereof) is positioned adjacent to a second expression cassette (e.g., an expression cassette encoding a second anti-VEGF agent, or a portion thereof). In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

In different embodiments, multicistronic expression constructs are provided in which the expression cassettes are oriented in different ways. For example, in some embodiments, a multicistronic expression construct is provided in which a first expression cassette is in the same orientation as a second expression cassette. In some embodiments, a multicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1—promoter2/encoding sequence 2-3′,

the expression cassettes are in the same orientation, the arrows indicate the direction of transcription of each of the cassettes. For another example, if a given nucleic acid construct comprises a sense strand comprising two expression cassettes in the configuration

5′-promoter 1/encoding sequence 1—encoding sequence 2/promoter 2-3′,

the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR, or in opposite orientation. AAV ITRs are directional. For example, the 3′ITR would be in the same orientation as the promoter1/encoding sequence 1 expression cassette of the examples above, but in opposite orientation to the 5′ITR, if both ITRs and the expression cassette would be on the same nucleic acid strand.

A large body of evidence suggests that multicistronic expression constructs often do not achieve optimal expression levels as compared to expression systems containing only one cistron. One of the suggested causes of sub-par expression levels achieved with multicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). Various strategies have been suggested to overcome the problem of promoter interference, for example, by producing multicistronic expression constructs comprising only one promoter driving transcription of multiple encoding nucleic acid sequences separated by internal ribosomal entry sites, or by separating cistrons comprising their own promoter with transcriptional insulator elements. All suggested strategies to overcome promoter interference are burdened with their own set of problems, though. For example, single-promoter driven expression of multiple cistrons usually results in uneven expression levels of the cistrons. Further some promoters cannot efficiently be isolated and isolation elements are not compatible with some gene transfer vectors, for example, some retroviral vectors.

In some embodiments of this invention, a multicistronic expression construct is provided that allows efficient expression of a first encoding nucleic acid sequence driven by a first promoter and of a second encoding nucleic acid sequence driven by a second promoter without the use of transcriptional insulator elements. Various configurations of such multicistronic expression constructs are provided herein, for example, expression constructs harboring a first expression cassette comprising an intron and a second expression cassette positioned within the intron, in either the same or opposite orientation as the first cassette. Other configurations are described in more detail elsewhere herein.

In some embodiments, multicistronic expression constructs are provided allowing for efficient expression of two or more encoding nucleic acid sequences. In some embodiments, the multicistronic expression construct comprises two expression cassettes. In some embodiments, a first expression cassette of a multicistronic expression construct as provided herein comprises a first RNA polymerase II promoter and a second expression cassette comprise a second RNA polymerase II promoter. In some embodiments, a first expression cassette of a multicistronic expression construct as provided herein comprises an RNA polymerase II promoter and a second expression cassette comprises an RNA polymerase III promoter.

In some embodiments, the multicistronic expression construct provided is a recombinant AAV (rAAV) construct.

In some embodiments, the isolated nucleic acid described herein comprises a codon optimized nucleic acid sequence of an anti-VEGF agent (e.g., KH902). Codon optimization of the nucleic acid coding sequence for optimized expression in target cells (e.g., mammalian cells) can be achieved by methods known in the art.

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some embodiments, isolated nucleic acid and rAAVs described herein comprise one or more of the following structural features (e.g., control or regulatory sequences): a long Chicken Beta Actin (CBA) promoter, an extended CBA intron, a Kozak sequence, an anti-VEGF agent (e.g., KH902) or codon-optimized anti-VEGF agent (e.g., KH902) variant-encoding nucleic acid sequence, one or more microRNA binding sites, and a rabbit beta-globin (RBG) poly A sequence. In some embodiments, one or more of the foregoing control sequences is operably linked to a nucleic acid sequence encoding an anti-VEGF agent (e.g., KH902).

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame.

In some embodiments, a transgene comprises a nucleic acid sequence encoding an anti-VEGF agent (e.g., KH902) operably linked to a promoter. A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

Generally, a promoter can be a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the chimeric cytomegalovirus chimeric cytomegalovirus (CMV)/Chicken β-actin (CB) promoter (CBA promotor), the SV40 promoter, the dihydrofolate reductase promoter, the β-actin promoter, the phosphoglycerol kinase (PGK) promoter, and the EF1α promoter [Invitrogen]. In some embodiments, a promoter is an RNA pol II promoter. In some embodiments, a promoter is the chimeric cytomegalovirus chimeric cytomegalovirus (CMV)/Chicken β-actin (CB) promoter (CBA promoter). In some embodiments, a promoter is an RNA pol III promoter, such as U6 or H1.

Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, the tissue-specific promoter is an eye-specific promoter. Examples of eye-specific promoters include retinoschisin proximal promoter, interphotoreceptor retinoid-binding protein enhancer (RS/IRBPa), rhodopsin kinase (RK), RPE65, and human cone opsin promoter.

In some embodiments, a promoter is a chicken beta-actin (CB) promoter. A chicken beta-actin promoter may be a short chicken beta-actin promoter or a long chicken beta-actin promoter. In some embodiments, a promoter (e.g., a chicken beta-actin promoter) comprises an enhancer sequence, for example a cytomegalovirus (CMV) enhancer sequence. A CMV enhancer sequence may be a short CMV enhancer sequence or a long CMV enhancer sequence. In some embodiments, a promoter comprises a long CMV enhancer sequence and a long chicken beta-actin promoter. In some embodiments, a promoter comprises a short CMV enhancer sequence and a short chicken beta-actin promoter. However, the skilled artisan recognizes that a short CMV enhancer may be used with a long CB promoter, and a long CMV enhancer may be used with a short CB promoter (and vice versa).

An isolated nucleic acid described herein may also contain one or more introns. In some embodiments, at least one intron is located between the promoter/enhancer sequence and the transgene. In some embodiments, an intron is a synthetic or artificial (e.g., heterologous) intron. Examples of synthetic introns include an intron sequence derived from SV-40 (referred to as the SV-40 T intron sequence) and intron sequences derived from chicken beta-actin gene. In some embodiments, a transgene described by the disclosure comprises one or more (1, 2, 3, 4, 5, or more) artificial introns. In some embodiments, the one or more artificial introns are positioned between a promoter and a nucleic acid sequence encoding an anti-VEGF agent (e.g., KH902).

In some embodiments, the transgene described herein comprises a Kozak sequence. A Kozak sequence is a nucleic acid motif comprising a consensus sequence GCC(A/G)CC (SEQ ID NO: 4) that is found in eukaryotic mRNA and plays a role in initiation of protein translation. In some embodiments, the Kozak sequence is positioned between the intron and the transgene encoding the anti-VEGF agent (e.g., KH902).

An isolated nucleic acid described by the disclosure may encode a transgene that further comprises a polyadenylation (poly A) sequence. In some embodiments, a transgene comprises a poly A sequence is a rabbit beta-globin (RBG) poly A sequence,

In some embodiments, the transgene comprises a 3′-untranslated region (3′-UTR). In some embodiments, the disclosure relates to isolated nucleic acids comprising a transgene encoding an anti-VEGF agent (e.g., KH902), and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in the 3′ untranslated region (3′-UTR) of a transgene, for example between the last codon of a nucleic acid sequence encoding an anti-VEGF agent (e.g., KH902), and a poly A sequence.

In some embodiments, a transgene comprises one or more (e.g., 1, 2, 3, 4, 5, or more) miRNA binding sites that de-target expression of anti-VEGF agent (e.g., KH902) from immune cells (e.g., antigen presenting cells (APCs), such as macrophages, dendrites, etc.). Incorporation of miRNA binding sites for immune-associated miRNAs may de-target transgene (e.g., KH902) expression from antigen presenting cells and thus reduce or eliminate immune responses (cellular and/or humoral) produced in the subject against products of the transgene, for example as described in US 2018/0066279, the entire contents of which are incorporated herein by reference.

In some aspects, the disclosure relates to isolated nucleic acids comprising a transgene encoding an anti-VEGF agent (e.g., KH902), and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more GM3S proteins, and a poly A sequence.

In some embodiments, a transgene comprises one or more (e.g., 1, 2, 3, 4, 5, or more) miRNA binding sites that de-target expression of the anti-VEGF agent (e.g., KH902) from liver cells. For example, in some embodiments, a transgene comprises one or more miR-122 binding sites.

In some embodiments, a transgene comprises one or more (e.g., 1, 2, 3, 4, 5, or more) miRNA binding sites that de-target expression of the one or more GM3S proteins from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune cell-associated miRNA” is a miRNA preferentially expressed in cells of the immune system, such as an antigen presenting cell (APC). In some embodiments, an immune cell-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune cell-associated miRNA is expressed is a B cell, T cell, Killer T cell, Helper T cell, γδ T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2 , CD1a, CD1d, CD11b (Mac-1), CD134 (OX40), CD150, CD2, CD25 (interleukin 2 receptor alpha), CD3, CD38, CD4, CD45RO, CDS, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune cell-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152. In some embodiments, a transgene described herein comprises one or more binding sites for miR-142.

In some embodiments, the isolated nucleic acid comprises inverted terminal repeats. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein (e.g., anti-VEGF agent such as KH902) and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are selected from the group consisting of AAV1 ITR, AAV2 ITR, AAV3 ITR, AAV4 ITR, AAVS ITR, and AAV6 ITR. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR, or ΔITR). Without wishing to be bound by any particular theory, a rAAV vector comprising an ITR lacking a functional TRS produces a self-complementary rAAV vector, for example as described by McCarthy (2008) Molecular Therapy 16(10):1648-1656. In some embodiments, vectors described herein comprise one or more AAV ITRs, and at least one ITR is an ITR variant of a known AAV serotype ITR. In some embodiments, the AAV ITR variant is a synthetic AAV ITR (e.g., AAV ITRs that do not occur naturally). In some embodiments, the AAV ITR variant is a hybrid ITR (e.g., a hybrid ITR comprises sequences derived from ITRs of two or more different AAV serotypes).

In some embodiments, an isolated nucleic acid (e.g., a rAAV vector) as described herein comprises, from 5′ to 3′ order: a 5′ AAV ITR, a CMV enhancer, a CBA promoter, an intron (e.g., chicken beta actin intron), a Kozak sequence, a transgene encoding an anti-VEGF agent (e.g., KH902) a rabbit beta-globin poly A, and a 3′ AAV ITR. An exemplary sequence of the isolated nucleic acid sequence is set forth in SEQ ID NO: 2. In some embodiments, the nucleic acid sequence comprises a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 2 (Kozak sequence underlined; KH902 coding sequence in bold):

(SEQ ID NO: 2)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGAAC
TATAGCTAGTCGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACCC
CCAATTTTGTATTTATTTATTTTTTAATTATTTTGTGCAGCGATGGGGGCGGGGGGGGGGGGGG
GGCGCGCGCCAGGCGGGGCGGGGCGGGGCGAGGGGCGGGGCGGGGCGAGGCGGAGAGGTGCGGC
GGCAGCCAATCAGAGCGGCGCGCTCCGAAAGTTTCCTTTTATGGCGAGGCGGCGGCGGCGGCGG
CCCTATAAAAAGCGAAGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTG
AGCGGGCGGGACGGCCCTTCTCCTCCGGGCTGTAATTAGCGCTTGGTTTAATGACGGCTTGTTT
CTTTTCTGTGGCTGCGTGAAAGCCTTGAGGGGCTCCGGGAGGGCCCTTTGTGCGGGGGGAGCGG
CTCGGGGGGTGCGTGCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGTGCGCGAGGGGAGCG
CGGCCGGGGGCGGTGCCCCGCGGTGCGGGGGGGGCTGCGAGGGGAACAAAGGCTGCGTGCGGGG
TGTGTGCGTGGGGGGGTGAGCAGGGGGTGTGGGCGCGTCGGTCGGGCTGCAACCCCCCCTGCAC
CCCCCTCCCCGAGTTGCTGAGCACGGCCCGGCTTCGGGTGCGGGGCTCCGTACGGGGCGTGGCG
CGGGGCTCGCCGTGCCGGGCGGGGGGTGGCGGCAGGTGGGGGTGCCGGGCGGGGCGGGGCCGCC
TCGGGCCGGGGAGGGCTCGGGGGAGGGGCGCGGCGGCCCCCGGAGCGCCGGCGGCTGTCGAGGC
GCGGCGAGCCGCAGCCATTGCCTTTTATGGTAATCGTGCGAGAGGGCGCAGGGACTTCCTTTGT
CCCAAATCTGTGCGGAGCCGAAATCTGGGAGGCGCCGCCGCACCCCCTCTAGCGGGCGCGGGGC
GAAGCGGTGCGGCGCCGGCAGGAAGGAAATGGGCGGGGAGGGCCTTCGTGCGTCGCCGCGCCGC
CGTCCCCTTCTCCCTCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCCTCTGCTAACCATGT
TCATGCCTTCTTCTTTTTCCTACAGCTCCTGGGCAACGTGCTGGTTATTGTGCTGTCTCATCAT
TTTGGCAAAGAATTCGCCACCATGGTCAGCTACTGGGACACCGGGGTCCTGCTGTGCGCGCTGC
TCAGCTGTCTGCTTCTCACAGGATCTAGTTCCGGAGGTAGACCTTTCGTAGAGATGTACAGTGA
AATCCCCGAAATTATACACATGACTGAAGGAAGGGAGCTCGTCATTCCCTGCCGGGTTACGTCA
CCTAACATCACTGTTACTTTAAAAAAGTTTCCACTTGACACTTTGATCCCTGATGGAAAACGCA
TAATCTGGGACAGTAGAAAGGGCTTCATCATATCAAATGCAACGTACAAAGAAATAGGGCTTCT
GACCTGTGAAGCAACAGTCAATGGGCATTTGTATAAGACAAACTATCTCACACATCGACAAACC
AATACAATCATAGATGTGGTTCTGAGTCCGTCTCATGGAATTGAACTATCTGTTGGAGAAAAGC
TTGTCTTAAATTGTACAGCAAGAACTGAACTAAATGTGGGGATTGACTTCAACTGGGAATACCC
TTCTTCGAAGCATCAGCATAAGAAACTTGTAAACCGAGACCTAAAAACCCAGTCTGGGAGTGAG
ATGAAGAAATTTTTGAGCACCTTAACTATAGATGGTGTAACCCGGAGTGACCAAGGATTGTACA
CCTGTGCAGCATCCAGTGGGCTGATGACCAAGAAGAACAGCACATTTGTCAGGGTCCATGAAAA
ACCTTTTGTTGCTTTTGGAAGTGGCATGGAATCTCTGGTGGAAGCCACGGTGGGGGAGCGTGTC
AGAATCCCTGCGAAGTACCTTGGTTACCCACCCCCAGAAATAAAATGGTATAAAAATGGAATAC
CCCTTGAGTCCAATCACACAATTAAAGCGGGGCATGTACTGACGATTATGGAAGTGAGTGAAAG
AGACACAGGAAATTACACTGTCATCCTTACCAATCCCATTTCAAAGGAGAAGCAGAGCCATGTG
GTCTCTCTGGTTGTGTATGTCCCACCGGGCCCGGGCGACAAAACTCACACATGCCCACTGTGCC
CAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGATGAGC
TGACCAAGAACCAGGTCAGCCTGACCTGCCTAGTCAAAGGCTTCTATCCCAGCGACATCGCCGT
GGAGTGGGAGAGCAATGGGCAGCCGGAGAACAACTACAAGGCCACGCCTCCCGTGCTGGACTCC
GACGGCTCCTTCTTCCTCTACAGCAAGCTCACCGTGGACAAGAGCAGGTGGCAGCAGGGGAACG
TCTTCTCATGCTCCGTGATGCATGAGGCTCTGCACAACCACTACACGCAGAAGAGCCTCTCCCT
GTCTCCGGGTAAATGAACGCGTGGTACCTCTAGAGTCGACCCGGGCGGCCTCGAGGACGGGGTG
AACTACGCCTGAGGATCCGATCTTTTTCCCTCTGCCAAAAATTATGGGGACATCATGAAGCCCC
TTGAGCATCTGACTTCTGGCTAATAAAGGAAATTTATTTTCATTGCAATAGTGTGTTGGAATTT
TTTGTGTCTCTCACTCGGAAGCAATTCGTTGATCTGAATTTCGACCACCCATAATACCCATTAC
CCTGGTAGATAAGTAGCATGGCGGGTTAATCATTAACTACAAGGAACCCCTAGTGATGGAGTTG
GCCACTCCCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAG

Also, within the scope of the present disclosure are vectors comprising the isolated nucleic acid described herein. An exemplary full vector sequence of pAAV-CBAOKH902 is set forth in SEQ ID NO: 3. In some embodiments, the vector comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 3:

(SEQ ID NO: 3)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGAAC
TATAGCTAGTCGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACCC
CCAATTTTGTATTTATTTATTTTTTAATTATTTTGTGCAGCGATGGGGGCGGGGGGGGGGGGGG
GGCGCGCGCCAGGCGGGGCGGGGCGGGGCGAGGGGCGGGGCGGGGCGAGGCGGAGAGGTGCGGC
GGCAGCCAATCAGAGCGGCGCGCTCCGAAAGTTTCCTTTTATGGCGAGGCGGCGGCGGCGGCGG
CCCTATAAAAAGCGAAGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTG
AGCGGGCGGGACGGCCCTTCTCCTCCGGGCTGTAATTAGCGCTTGGTTTAATGACGGCTTGTTT
CTTTTCTGTGGCTGCGTGAAAGCCTTGAGGGGCTCCGGGAGGGCCCTTTGTGCGGGGGGAGCGG
CTCGGGGGGTGCGTGCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGTGCGCGAGGGGAGCG
CGGCCGGGGGCGGTGCCCCGCGGTGCGGGGGGGGCTGCGAGGGGAACAAAGGCTGCGTGCGGGG
TGTGTGCGTGGGGGGGTGAGCAGGGGGTGTGGGCGCGTCGGTCGGGCTGCAACCCCCCCTGCAC
CCCCCTCCCCGAGTTGCTGAGCACGGCCCGGCTTCGGGTGCGGGGCTCCGTACGGGGCGTGGCG
CGGGGCTCGCCGTGCCGGGCGGGGGGTGGCGGCAGGTGGGGGTGCCGGGCGGGGCGGGGCCGCC
TCGGGCCGGGGAGGGCTCGGGGGAGGGGCGCGGCGGCCCCCGGAGCGCCGGCGGCTGTCGAGGC
GCGGCGAGCCGCAGCCATTGCCTTTTATGGTAATCGTGCGAGAGGGCGCAGGGACTTCCTTTGT
CCCAAATCTGTGCGGAGCCGAAATCTGGGAGGCGCCGCCGCACCCCCTCTAGCGGGCGCGGGGC
GAAGCGGTGCGGCGCCGGCAGGAAGGAAATGGGCGGGGAGGGCCTTCGTGCGTCGCCGCGCCGC
CGTCCCCTTCTCCCTCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCCTCTGCTAACCATGT
TCATGCCTTCTTCTTTTTCCTACAGCTCCTGGGCAACGTGCTGGTTATTGTGCTGTCTCATCAT
TTTGGCAAAGAATTCGCCACCATGGTCAGCTACTGGGACACCGGGGTCCTGCTGTGCGCGCTGC
TCAGCTGTCTGCTTCTCACAGGATCTAGTTCCGGAGGTAGACCTTTCGTAGAGATGTACAGTGA
AATCCCCGAAATTATACACATGACTGAAGGAAGGGAGCTCGTCATTCCCTGCCGGGTTACGTCA
CCTAACATCACTGTTACTTTAAAAAAGTTTCCACTTGACACTTTGATCCCTGATGGAAAACGCA
TAATCTGGGACAGTAGAAAGGGCTTCATCATATCAAATGCAACGTACAAAGAAATAGGGCTTCT
GACCTGTGAAGCAACAGTCAATGGGCATTTGTATAAGACAAACTATCTCACACATCGACAAACC
AATACAATCATAGATGTGGTTCTGAGTCCGTCTCATGGAATTGAACTATCTGTTGGAGAAAAGC
TTGTCTTAAATTGTACAGCAAGAACTGAACTAAATGTGGGGATTGACTTCAACTGGGAATACCC
TTCTTCGAAGCATCAGCATAAGAAACTTGTAAACCGAGACCTAAAAACCCAGTCTGGGAGTGAG
ATGAAGAAATTTTTGAGCACCTTAACTATAGATGGTGTAACCCGGAGTGACCAAGGATTGTACA
CCTGTGCAGCATCCAGTGGGCTGATGACCAAGAAGAACAGCACATTTGTCAGGGTCCATGAAAA
ACCTTTTGTTGCTTTTGGAAGTGGCATGGAATCTCTGGTGGAAGCCACGGTGGGGGAGCGTGTC
AGAATCCCTGCGAAGTACCTTGGTTACCCACCCCCAGAAATAAAATGGTATAAAAATGGAATAC
CCCTTGAGTCCAATCACACAATTAAAGCGGGGCATGTACTGACGATTATGGAAGTGAGTGAAAG
AGACACAGGAAATTACACTGTCATCCTTACCAATCCCATTTCAAAGGAGAAGCAGAGCCATGTG
GTCTCTCTGGTTGTGTATGTCCCACCGGGCCCGGGCGACAAAACTCACACATGCCCACTGTGCC
CAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGATGAGC
TGACCAAGAACCAGGTCAGCCTGACCTGCCTAGTCAAAGGCTTCTATCCCAGCGACATCGCCGT
GGAGTGGGAGAGCAATGGGCAGCCGGAGAACAACTACAAGGCCACGCCTCCCGTGCTGGACTCC
GACGGCTCCTTCTTCCTCTACAGCAAGCTCACCGTGGACAAGAGCAGGTGGCAGCAGGGGAACG
TCTTCTCATGCTCCGTGATGCATGAGGCTCTGCACAACCACTACACGCAGAAGAGCCTCTCCCT
GTCTCCGGGTAAATGAACGCGTGGTACCTCTAGAGTCGACCCGGGCGGCCTCGAGGACGGGGTG
AACTACGCCTGAGGATCCGATCTTTTTCCCTCTGCCAAAAATTATGGGGACATCATGAAGCCCC
TTGAGCATCTGACTTCTGGCTAATAAAGGAAATTTATTTTCATTGCAATAGTGTGTTGGAATTT
TTTGTGTCTCTCACTCGGAAGCAATTCGTTGATCTGAATTTCGACCACCCATAATACCCATTAC
CCTGGTAGATAAGTAGCATGGCGGGTTAATCATTAACTACAAGGAACCCCTAGTGATGGAGTTG
GCCACTCCCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCCTTAATTAACCTAATTCACT
GGCCGTCGTTTTACAACGTCGTGACTGGGAAAACCCTGGCGTTACCCAACTTAATCGCCTTGCA
GCACATCCCCCTTTCGCCAGCTGGCGTAATAGCGAAGAGGCCCGCACCGATCGCCCTTCCCAAC
AGTTGCGCAGCCTGAATGGCGAATGGGACGCGCCCTGTAGCGGCGCATTAAGCGCGGCGGGTGT
GGTGGTTACGCGCAGCGTGACCGCTACACTTGCCAGCGCCCTAGCGCCCGCTCCTTTCGCTTTC
TTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGATGGTTC
ACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACCCTATCTCGGTCTATTCTTTTGATT
TATAAGGGATTTTGCCGATTTCGGCCTATTGGTTAAAAAATGAGCTGATTTAACAAAAATTTAA
CGCGAATTTTAACAAAATATTAACGCTTACAATTTAGGTGGCACTTTTCGGGGAAATGTGCGCG
GAACCCCTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAAAGTTCTG
CTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTCTAGCTTCC
CGGCAACAATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTACATACCTCG
CTCTGCTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCGCTCGCCGC
AGCCGAACGACCGAGCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGGTTTCCCGACTGGAA
AGCGGGCAGTGAGCGCAACGCAATTAATGTGAGTTAGCTCACTCATTAGGCACCCCAGGCTTTA
CACTTTATGCTTCCGGCTCGTATGTTGTGTGGAATTGTGAGCGGATAACAATTTCACACAGGAA
ACAGCTATGACCATGATTACGCCAGATTTAATTAAGGCCTTAATTAGG

In some embodiments, the anti-VEGF agent (e.g., KH902) described herein can be delivered to a subject via a non-viral platform. In some embodiments, the anti-VEGF agent (e.g., KH902) described herein can be delivered to a subject via closed-ended linear duplex DNA (ceDNA). Delivery of a transgene (e.g., anti-VEGF agent such as KH902) has been described previously, see e.g., WO2017152149, the entire contents of which are incorporated herein by reference. In some embodiments, the nucleic acids having asymmetric terminal sequences (e.g., asymmetric interrupted self-complementary sequences) form closed-ended linear duplex DNA structures (e.g., ceDNA) that, in some embodiments, exhibit reduced immunogenicity compared to currently available gene delivery vectors. In some embodiments, ceDNA behaves as linear duplex DNA under native conditions and transforms into single-stranded circular DNA under denaturing conditions. Without wishing to be bound by any particular theory, ceDNA are useful, in some embodiments, for the delivery of a transgene (e.g., anti-VEGF agent such as KH902) to a subject.
Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., ocular tissues). The AAV capsid is an important element in determining these tissue-specific targeting capabilities (e.g., tissue tropism). Thus, an rAAV having a capsid appropriate for the tissue being targeted can be selected.

In some embodiments, the rAAV of the present disclosure comprises a capsid protein containing the isolated nucleic acid described herein. In some embodiments, rAAVs of the disclosure comprise a nucleotide sequence as set forth in SEQ ID NO: 2. In some embodiments, rAAVs of the disclosure contains a nucleotide sequence that is 99% identical, 95% identical, 90% identical, 85% identical, 80% identical, 75% identical, 70% identical, 65% identical, 60% identical, 55% identical, or 50% identical to a nucleotide sequence as set forth in SEQ ID NO: 2.

Methods for obtaining recombinant AAVs having a desired capsid protein are well known in the art. (See, for example, US 2003/0138772, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for ocular tissues or muscle tissue. In some embodiments, an AAV capsid protein targets ocular cell types (e.g., cornea, photoreceptor cells, retinal cells, etc.).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP, and variants of any of the foregoing. In some embodiments, an AAV capsid protein is of a serotype derived from a non-human primate, for example AAVrh8 serotype. In some embodiments, the capsid protein is of AAV serotype 6 (e.g., AAV6 capsid protein), AAV serotype 8 (e.g., AAV8 capsid protein), AAV serotype 2 (e.g., AAV2 capsid protein), AAV serotype 5 (e.g., AAV5 capsid protein), or AAV serotype 9 (e.g., AAV9 capsid protein). In some embodiments, the AAV capsid is AAV1.

In some embodiments, the AAV capsid is AAV2. In some embodiments, the AAV capsid protein with desired tissue tropism can be selected from AAV capsid proteins isolated from mammals (e.g., tissue from a subject).

In some embodiments, the rAAV described herein is a single stranded AAV (ssAAV). An ssAAV, as used herein, refers to an rAAV with the coding sequence and complementary sequence of the transgene expression cassette on separate strands and are packaged in separate viral capsids.

The components to be cultured in the host cell to package an rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence encoding a transgene (e.g., KH902). A “host cell” refers to any cell that harbors, or is capable of harboring, a substance of interest. Often a host cell is a mammalian cell. In some embodiments, a host cell is a photoreceptor cell, retinal pigment epithelial cell, keratinocyte, corneal cell, and/or a tumor cell. A host cell may be used as a recipient of an AAV helper construct, an AAV vector, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a neuron, a photoreceptor cell, a pigmented retinal epithelial cell, or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, an anellovirus vector (e.g., Anellovirus vector as described in US20200188456A1), etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

### AAV-mediated Delivery of a Transgene to Ocular Tissue

Aspects of the instant disclosure relate to compositions comprising a recombinant AAV comprising a capsid protein and a nucleic acid encoding a transgene, wherein the transgene comprises a nucleic acid sequence encoding an anti-VEGF agent (e.g., KH902). In some embodiments, the nucleic acid further comprises AAV ITRs.

The isolated nucleic acids, vectors, rAAVs, and compositions comprising the isolated nucleic acid described herein, the vectors described herein, or the rAAV described herein of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, the subject is a human.

In some embodiments, administration of an isolated nucleic and/or an rAAV as described herein result in delivery of the transgene (e.g., KH902) to ocular tissue. Delivery of the rAAVs to a mammalian subject may be by, for example, intraocular injection, subretinal injection, topical administration (e.g., an eye drop), or by injection into the eye of the mammalian subject to ocular tissues (e.g., intravitreal injection). As used herein, “ocular tissues” refers to any tissue derived from or contained in the eye. Non-limiting examples of ocular tissues include neurons, retina (e.g., photoreceptor cells), sclera, choroid, retina, vitreous body, macula, fovea, optic disc, lens, pupil, iris, aqueous fluid, cornea (e.g., keratocytes, corneal endothelial cells, corneal basal cells, corneal wing cells, and corneal squamous cells), conjunctiva ciliary body, and optic nerve. The retina is located in the posterior of the eye and comprises photoreceptor cells. These photoreceptor cells (e.g., rods, cones) confer visual acuity by discerning color, as well as contrast in the visual field.

Alternatively, delivery of the rAAVs to a mammalian subject may be by intramuscular injection or by administration into the bloodstream of the mammalian subject. Administration into the bloodstream may be by injection into a vein, an artery, or any other vascular conduit. Non-limiting exemplary methods of intramuscular administration of the rAAV include Intramuscular (IM) Injection and Intravascular Limb Infusion. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. In some embodiments, an rAAV or a composition (e.g., composition containing the isolated nucleic acid or the rAAV) as described in the disclosure is administered by intravitreal injection. In some embodiments, an rAAV or a composition (e.g., composition containing the isolated nucleic acid or the rAAV) as described in the disclosure is administered by intraocular injection. In some embodiments, an rAAV or a composition (e.g., composition containing the isolated nucleic acid or the rAAV) as described in the disclosure is administered by subretinal injection. In some embodiments, an rAAV or a composition (e.g., composition containing the isolated nucleic acid or the rAAV) as described in the disclosure is administered by intravenous injection. In some embodiments, an rAAV or a composition (e.g., composition containing the isolated nucleic acid or the rAAV) as described in the disclosure is administered by intramuscular injection. In some embodiments, an rAAV or a composition (e.g., composition containing the isolated nucleic acid or the rAAV) as described in the disclosure is administered by intratumoral injection.

In some embodiments, administration of an isolated nucleic and/or an rAAV as described herein results in inhibition of VEGF (e.g., VEGF activity). In some embodiments, administration of an isolated nucleic acid and/or an rAAV as described herein results in inhibition of VEGF (e.g., VEGF activity) in ocular tissue. The extent of VEGF inhibition can be measured by any suitable known method (e.g., HUVEC angiogenesis assay, retinal vascular development assay, retinal edema assay, laser damage-induced choroidal neovascular (CNVs), etc.). In some embodiments, VEGF (e.g., VEGF activity) in subjects received anti-VEGF agent (e.g., injected with an isolated nucleic acid and/or a rAAV described herein) is inhibited by at least 2%, at least 5%, at least 10%, at least 15%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 100% compared to an uninjected subject, or the same subject before receiving the anti-VEGF agent. In some embodiments, the VEGF (e.g., VEGF activity) in an uninjected subject, or a subject prior to receiving an anti-VEGF agent is by at least 2%, at least 5%, at least 10%, at least 15%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 100%, at least 1-fold, at least 2-fold at least 3-fold, at least 4-fold, at least 5-fold, at least 6-fold, at least 7-fold, at least 8-fold, at least 9-fold, at least 10-fold, at least 10 to 50-fold (e.g., 10-fold, 20-fold, 30-fold, 40-fold, or 50-fold), at least 50 to 100-fold (e.g., 50-fold, 60-fold, 70-fold, 80-fold, 90-fold or 100-fold) or more higher compared to a subject received anti-VEGF agent administration (e.g., injected with an isolated nucleic acid and/or a rAAV described herein). In some embodiments, administration of an anti-VEGF agent (e.g., an isolated nucleic acid and/or a rAAV described herein) result in inhibition of VEGF (e.g., VEGF activity) for longer than 1 day, longer than 2 days, longer than 3 days, longer than 4 days, longer than 5 days, longer than 6 days, longer than 7 days, longer than 1 week (e.g., 8 days, 9 days, 10 days, 11 days, 12 days, 13 days, or 14 days), longer than 2 weeks (e.g., 15 days, 16 days, 17 days, 18 days, 19 days, 20 days, or 21 days), longer than 3 weeks week (e.g., 22 days, 23 days, 24 days, 25 days, 25 days, 27 days, or 28 days), longer than 4 weeks (e.g., 29 days, 30 days, 40 days, 50 days, 60 days, 100 days or more), longer than 1 month (e.g., 5 weeks, 6 weeks, 7 weeks, 8 weeks, 9 weeks, 10 weeks, or more), longer than 2 months (e.g., between 2 months to 2.5 months, between 2 months to 3 months, between 2 months to 4 months, between 2 months to 5 months, between 2 months to 6 months, between 2 months to 7 months, between 2 months to 8 months, between 2 months to 9 months, between 2 months to 10 months, between 2 months to 11 months, between 2 months to 12 months), longer than 3 months (e.g., between 3 months to 4 months, between 3 months to 5 months, between 3 months to 6 months, between 3 months to 7 months, between 3 months to 8 months, between 3 months to 9 months, between 3 months to 10 months, between 3 months to 11 months, between 3 months to 12 months), longer than 4 months (e.g., between 4 months to 5 months, between 4 months to 6 months, between 4 months to 7 months, between 4 months to 8 months, between 4 months to 9 months, between 4 months to 10 months, between 4 months to 11 months, between 4 months to 12 months), longer than 5 months (e.g., between 5 months to 6 months, between 5 months to 7 months, between 5 months to 8 months, between 5 months to 9 months, between 5 months to 10 months, between 5 months to 11 months, between 5 months to 12 months), longer than 6 months (e.g., between 6 months to 7 months, between 6 months to 8 months, between 6 months to 9 months, between 6 months to 10 months, between 6 months to 11 months, between 6 months to 12 months), longer than 7 months (e.g., between 7 months to 8 months, between 7 months to 9 months, between 7 months to 10 months, between 7 months to 11 months, between 7 months to 12 months), longer than 8 months (e.g., between 8 months to 9 months, between 8 months to 10 months, between 8 months to 11 months, between 8 months to 12 months), longer than 9 months (e.g., between 9 months to 10 months, between 9 months to 11 months, between 9 months to 12 months), longer than 10 months (e.g., between 10 months to 11 months, between 11 months to 12 months), longer than 11 months (e.g., between 11 months to 12 months), longer than 12 months (e.g., 12 to 15 months, 12-18 months, 12-21 months, 12-2 months), longer than 1 year (e.g., 1 to 1.5 years), longer than 2 year, longer than 3 year, longer than 4 year, longer than 5 year, longer than 10 years, longer than 15 years, longer than 20 years or more.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs or the compositions (e.g., composition containing the isolated nucleic acid or the rAAV described herein) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intravitreal delivery to the eye), intraocular injection, subretinal injection, oral, inhalation (including intranasal and intratracheal delivery), intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

An effective amount of rAAVs or composition (e.g., composition containing the isolated nucleic acid or the rAAV described herein) is an amount sufficient to target infect an animal, target a desired tissue (e.g., muscle tissue, ocular tissue, etc.). In some embodiments, an effective amount of an rAAV is administered to the subject during a pre-symptomatic stage of degenerative disease. In some embodiments, a subject is administered an rAAV or composition after exhibiting one or more signs or symptoms of degenerative disease. In some embodiments, the effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1 x 10¹⁶, inclusive). In some embodiments, an effective amount of an rAAV ranges between 1×10⁹ and 1×10¹⁴ genome copies of the rAAV. In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of about 10¹² to 10¹⁴ rAAV genome copies is appropriate. In some embodiments, a dosage of about 10¹³ to 10¹⁴ rAAV genome copies is appropriate. In some embodiments, a dosage of about 1×10¹², about 1.1×10¹², about 1.2×10¹², about 1.3×10¹², about 1.4×10¹², about 1.5×10¹², about 1.6×10¹², about 1.7×10¹², about 1.8×10¹², about 1.9×10¹², about 1×10¹³, about 1.1×10¹³, about 1.2×10¹³, about 1.3×10¹³, about 1.4×10¹³, about 1.5×10¹³, about 1.6×10¹³, about 1.7×10¹³, about 1.8×10¹³, about 1.9×10¹³, or about 2.0×10¹⁴ vector genome (vg) copies per kilogram (kg) of body weight is appropriate. In some embodiments, a dosage of between about 4×10¹² to 2×10¹³ rAAV genome copies is appropriate. In some embodiments a dosage of about 1.5×10¹³ vg/kg by intravenous administration is appropriate. In certain embodiments, 10¹²-10¹³ rAAV genome copies is effective to target tissues (e.g., the eye). In certain embodiments, 10¹³-10¹⁴ rAAV genome copies is effective to target tissues effective to target tissues (e.g., the eye).

In some embodiments, the rAAV is injected into the subject. In other embodiments, the rAAV is administrated to the subject by topical administration (e.g., an eye drop). In some embodiments, an effective amount of an rAAV is the amount sufficient to express an effective amount of the anti-VEGF agent (e.g., KH902) in the target tissue (e.g., the eyes) of a subject.

In some embodiments, delivery of an effective amount of rAAV by injection (e.g., delivering an rAAV encoding an anti-VEGF agent (e.g., KH902) is in an amount such that it is sufficient to express an effective amount of an anti-VEGF agent (e.g., KH902) in the target tissue). In some embodiments, delivery of an effective amount of an rAAV encoding an anti-VEGF agent (e.g., KH902) is sufficient to deliver 10 μg to 10 mg of an anti-VEGF agent (e.g., KH902) or any intermediate value in between to the subject per eye by suitable routes of administration (e.g., intraocular injection, i.v. injection, intraperitoneal injection and intramuscular injection. In some embodiments, the rAAV encoding an anti-VEGF agent (e.g., KH902) is sufficient to deliver 20 μg to 5 mg or any intermediate value in between of an anti-VEGF agent (e.g., KH902) to the subject per eye. In some embodiments, the rAAV encoding an anti-VEGF agent (e.g., KH902) is sufficient to deliver 10 μg, 20 μg, 30 μg, 40 μg, 50 μg, 60 μg, 70 μg, 80 μg, 90 μg, 100 μg, 200 μg, 300 μg, 400 μg, 500 μg, 600 μg, 700 μg, 800 μg, 900 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg or more of an anti-VEGF agent (e.g., KH902) to the subject per eye.

In some embodiments, the rAAV encoding an anti-VEGF agent (e.g., KH902) is administered to the subject once a day, once a week, once every two weeks, once a month, once every 2 months, once every 3 months, once every 6 months, once a year, or once in a lifetime of the subject.

In some embodiments, delivery of an effective amount of rAAV by topical administration such as an eye drop (e.g., delivering an rAAV encoding an anti-VEGF agent (e.g., KH902) is in an amount such that it is sufficient to express an effective amount of an anti-VEGF agent (e.g., KH902) in the target tissue). In some embodiments, the eye drop containing the rAAV encoding is administered to the subject once a week, once a month, once every 3 months, once every 6 months, or once a year.

In some embodiments, the eye drop comprises the rAAV encoding an anti-VEGF agent (e.g., KH902) sufficient to deliver the anti-VEGF agent at a concentration of 1 mg/ml to 20 mg/ml. In some embodiments, the eye drop comprises the rAAV encoding an anti-VEGF agent (e.g., KH902) sufficient to deliver the anti-VEGF agent at a concentration of 2.5 mg/ml to 10 mg/ml. In some embodiments, the eye drop comprises the rAAV encoding an anti-VEGF agent (e.g., KH902) sufficient to deliver the anti-VEGF agent at a concentration of 1 mg/ml, 2 mg/ml, 2.5 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, or 20 mg/ml. In some embodiments, the eye drop is administered at 0.01 ml, 0.02 ml, 0.03 ml, 0.04 ml, 0.05 ml, 0.06 ml, 0.07 ml, 0.08 ml, 0.09 ml, 0.1 ml, 0.2 ml, 0.3 ml, 0.4 ml or 0.5 ml.

An effective amount of rAAVs or composition (e.g., composition containing the isolated nucleic acid or the rAAV described herein) may also depend on the mode of administration. For example, targeting an ocular (e.g., corneal) tissue by intrastromal administration or subcutaneous injection may require different (e.g., higher or lower) doses, in some cases, than targeting an ocular (e.g., corneal) tissue by another method (e.g., systemic administration, topical administration). In some embodiments, intrastromal injection (IS) of rAAV having certain serotypes (e.g., AAV2, AAVS, AAV6, AAV6.2, AAV7, AAV8, AAV9, AAVrh.8, AAVrh.10, AAVrh.39, and AAVrh.43) mediates efficient transduction of ocular (e.g., corneal, retinal, etc.) cells. Thus, in some embodiments, the injection is intrastromal injection (IS). In some embodiments, the injection is topical administration (e.g., topical administration to an eye). In some cases, multiple doses of a rAAV are administered.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions are well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf-life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intravitreally, intraocularly, subretinally, subcutaneously, intrapancreatically, intranasally, parenterally, intravenously, intramuscularly, intrathecally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that it is easily syringed. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the anti-VEGF agent described herein (e.g., KH902) is delivered to the subject by ceDNA. Any compositions containing ceDNA encoding the anti-VEGF agent (e.g., KH902) are also within the scope of the present disclosure. In some embodiments, the ceDNA encoding the anti-VEGF agent (e.g., KH902) and the compositions thereof can be administered to the subject using any suitable method described herein. In some embodiments, delivery of an effective amount of the ceDNA encoding the anti-VEGF agent (e.g., KH902) by injection is in an amount such that it is sufficient to express an effective amount of an anti-VEGF agent (e.g., KH902) in the target tissue). In some embodiments, delivery of an effective amount of a ceDNA encoding the anti-VEGF agent (e.g., KH902) is sufficient to deliver 10 μg to 10 mg of an anti-VEGF agent (e.g., KH902) or any intermediate value in between to the subject per eye by suitable routes of administration (e.g., intraocular injection, i.v. injection, intraperitoneal injection and intramuscular injection. In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, Solu-Medrol, Lansoprazole, trimethoprim/sulfamethoxazole, methotrexate, and any combination thereof. In some embodiments, the immunosuppression regimen comprises administering sirolimus, prednisolone, lansoprazole, trimethoprim/sulfamethoxazole, or any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, the methods described in this disclosure further comprise co-administration or prior administration of an agent to a subject administered an rAAV or pharmaceutical composition comprising an rAAV of the disclosure. In some embodiments, the agent is selected from a group consisting of Miglustat, Keppra, Prevacid, Clonazepam, and any combination thereof. In some embodiments, the rAAV (e.g., rAAV for KH902) and the additional agent can be delivered to the subject in any order. In some embodiments, the rAAV (e.g., rAAV for KH902) and the additional agent (e.g., Miglustat, Keppra, Prevacid, Clonazepam) are delivered to the subject simultaneously. In some embodiments, the rAAV (e.g., rAAV for KH902) and the additional agent (e.g., Miglustat, Keppra, Prevacid, Clonazepam) are co-administered to the subject (e.g., in one composition or in different compositions). In some embodiments, the rAAV (e.g., rAAV for KH902) is delivered before the additional agent (e.g., Miglustat, Keppra, Prevacid, Clonazepam). In some embodiments, the rAAV (e.g., rAAV for KH902) is delivered after the additional agent (e.g., Miglustat, Keppra, Prevacid, Clonazepam). In some embodiments, the rAAV (e.g., rAAV for KH902) and the additional agent (e.g., Miglustat, Keppra, Prevacid, Clonazepam) are delivered to the subject at different frequencies, for example, the subject receives the rAAV (e.g., rAAV for KH902) every month, every two-months, every six-months, every year, every two years, every three years, every 5 years, or longer, but receives the additional agent (e.g., Miglustat, Keppra, Prevacid, Clonazepam) daily, weekly, biweekly, monthly, twice a day, three times a day, or twice a week, etc.

In some embodiments, immunosuppression of a subject maintained during and/or after administration of a rAAV or pharmaceutical composition. In some embodiments, a subject is immunosuppressed (e.g., administered one or more immunosuppressants) for between 1 day and 1 year after administration of the rAAV or pharmaceutical composition.

Methods of Treating Diseases Associated with VEGF and/or Angiogenesis

Aspects of the disclosure relate to methods for delivering a transgene encoding an anti-VEGF agent (e.g., KH902) to a subject (e.g., a cell in a subject). In some embodiments, the subject is a human. In some embodiments, the subject is a non-human mammal. Non-limiting examples of non-human mammals are mouse, rat, cat, dog, sheep, rabbit, horse, cow, goat, pig, guinea pig, hamster, chicken, turkey, or a non-human primate.

In some embodiments, the present disclosure relates to a method for inhibiting VEGF activity in a subject in need thereof. In some embodiments, methods described by the disclosure are useful for treating a subject having or suspected of having a disease associated with VEGF. As used herein, “VEGF-associated diseases” refers to set of diseases associated with aberrant VEGF activity/signaling. VEGF is a signal protein produced by cells that stimulates the formation of blood vessels. VEGF is a known factor to induce angiogenesis. In some embodiments, methods described by the disclosure are useful for treating a subject having or suspected of having an angiogenesis associated disease. An angiogenesis associated disease, as used herein, refers to diseases related to abnormal angiogenesis. Non-limiting exemplary angiogenesis associated diseases include angiogenesis-dependent cancer, including, for example, angiogenesis associated eye diseases, solid tumors (e.g., lung cancer, breast cancer, kidney cancer, liver cancer, pancreatic cancer, head and neck cancer, colon cancer, melanoma), blood born tumors such as leukemias, metastatic tumors, benign tumors (e.g., hemangiomas, acoustic neuromas, neurofibromas, trachomas, and pyogenic granulomas), rheumatoid arthritis, psoriasis, rubeosis, Osier-Webber Syndrome, myocardial angiogenesis, plaque neovascularization, telangiectasia, hemophiliac joints, or angiofibroma.

In some embodiments, angiogenesis-associated eye diseases include but are not limited to diabetic retinopathy, retinopathy of prematurity, macular degeneration, corneal graft rejection, neovascular glaucoma, and retrolental fibroplasias, epidemic keratoconjunctivitis, Vitamin A deficiency, contact lens overwear, atopic keratitis, superior limbic keratitis, pterygium keratitis sicca, Sjogren's, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren's ulcer, Terrien's marginal degeneration, marginal keratolysis, rheumatoid arthritis, systemic lupus, polyarteritis, trauma, Wegener's sarcoidosis, Scleritis, Steven's Johnson disease, pemphigoid radial keratotomy, and corneal graft rejection, sickle cell anemia, sarcoid, pseudoxanthoma elasticum, Paget's disease, vein occlusion, artery occlusion, carotid obstructive disease, chronic uveitis/vitritis, mycobacterial infections, Lyme's disease, systemic lupus erythematosus, retinopathy of prematurity, Eales disease, Behcet's disease, infections causing a retinitis or choroiditis, presumed ocular histoplasmosis, Bests disease, myopia, optic pits, Stargardt disease, pars planitis, chronic retinal detachment, hyperviscosity syndromes, toxoplasmosis, trauma or post-laser complications.

As used herein, the term “treating” refers to the application or administration of a composition comprising an anti-VEGF agent (e.g., KH902) to a subject, who has a symptom or a disease associated with aberrant VEGF activity or angiogenesis, or a predisposition toward a disease associated with aberrant VEGF activity or angiogenesis, with the purpose to cure, heal, alleviate, relieve, alter, remedy, ameliorate, improve, or affect the disorder, the symptom of the disease, or the predisposition toward a disease associated with aberrant VEGF activity or angiogenesis. In some embodiments, administration of an anti-VEGF agent results in a reduction of VEGF activity by 2%, 5%, 10%, 20%, 30%, 40%, 50%, 60%, 70%, 80%, 90%, or 100% compared to a reference value. Methods of measuring VEGF activity are known in the art. Non-limiting exemplary reference value can be VEGF activity of the same subject prior to receiving anti-VEGF agent treatment. In some embodiments, administration of an anti-VEGF agent results in a reduction of angiogenesis by 2%, 5%, 10%, 20%, 30%, 40%, 50%, 60%, 70%, 80%, 90%, or 100% compared to a reference value. Methods of measuring angiogenesis are known in the art. Non-limiting exemplary reference value can be level of angiogenesis of the same subject prior to receiving anti-VEGF agent treatment.

Alleviating a disease associated with aberrant VEGF activity or angiogenesis includes delaying the development or progression of the disease or reducing disease severity. Alleviating the disease does not necessarily require curative results. As used therein, “delaying” the development of a disease (such as a disease associated with aberrant VEGF activity or angiogenesis) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with aberrant VEGF activity or angiogenesis includes initial onset and/or recurrence.

### EXAMPLES

### Example 1: A rAAV Vector Platform to Deliver Conbercept (KH902)

An rAAV vector platform to deliver Conbercept (KH902), an anti-VEGF therapeutic agent into the retina via intravitreal administration (or other routes) was described here. The unique design is a single-strand AAV vector genome that contains the KH902 transgene driven by the CMV enhancer/chicken (3-actin promoter regulatory cassette (FIGS. 1A-1C). A Kozak sequence was designed upstream of the KH902 start codon to enhance translation (FIG. 1C). When the cis-plasmid (FIG. 1A) was delivered into packaging cell lines that expressed the AAV Rep and Cap genes and obligatory helper genes via trans-plasmid co-transfections or by stable integration, sequences that include and are flanked by the inverted terminal repeat sequences (ITRs) were packaged into AAV capsid virions.

Cells were infected or transduced by the resulting ssAAV-KH902 virions expressed secreted KH902, which was detected by standard Western blot analysis (FIG. 2). Transduction of retinal pigment epithelial (RPE) cell lines with ssAAV-KH902 packaged into AAV2 capsid, resulted in protein expression of KH902 with similar molecular weights as the Conbercept drug, which was produced in Chinese Hamster Ovary (CHO) cell lines. This data indicates that the rAAV-KH902 vector can be packaged into different AAV capsids and when infected into cells can secrete KH902 effectively.

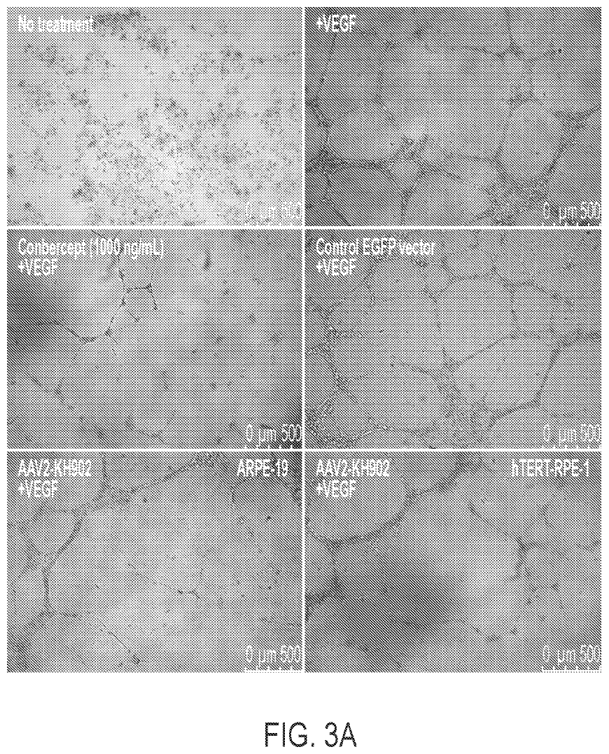

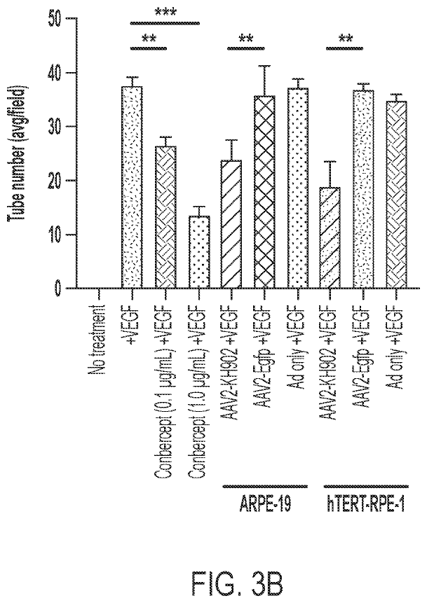

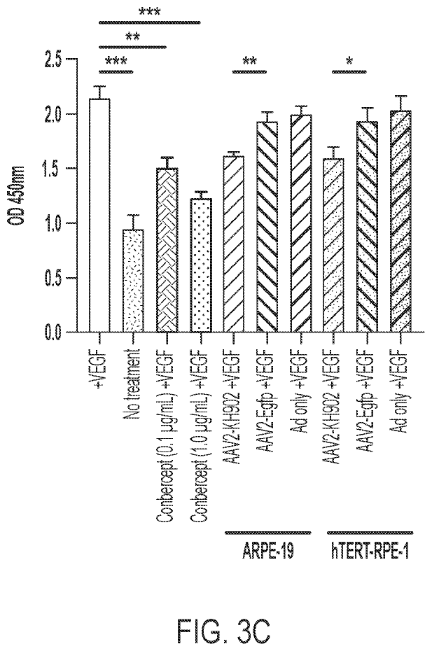

Conditioned media from RPE cells infected with rAAV-KH902 robustly inhibited angiogenesis as indicated by a reduction in vascular endothelial growth factor (VEGF)-induced tubulogenesis (FIG. 3A and 3B) and proliferation (CCK-8, FIG. 3C) of human umbilical vein endothelial cells (HUVECs) in the same fashion as the Conbercept drug. This data suggest that cells infected with rAAV-KH902 can express and secrete functional anti-angiogenic KH902 in vitro.

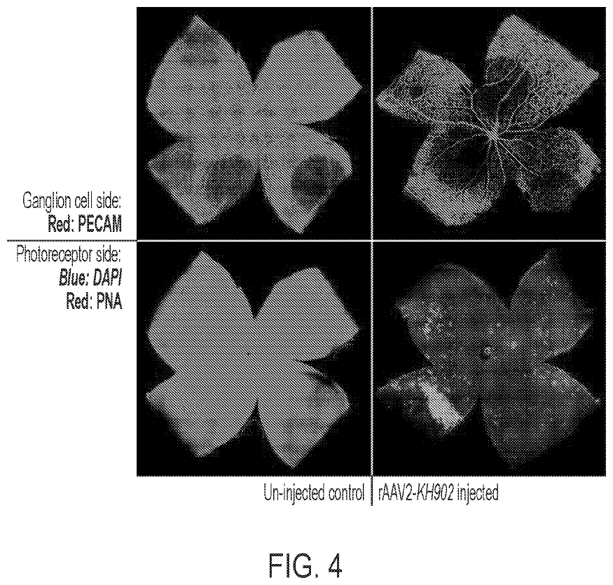

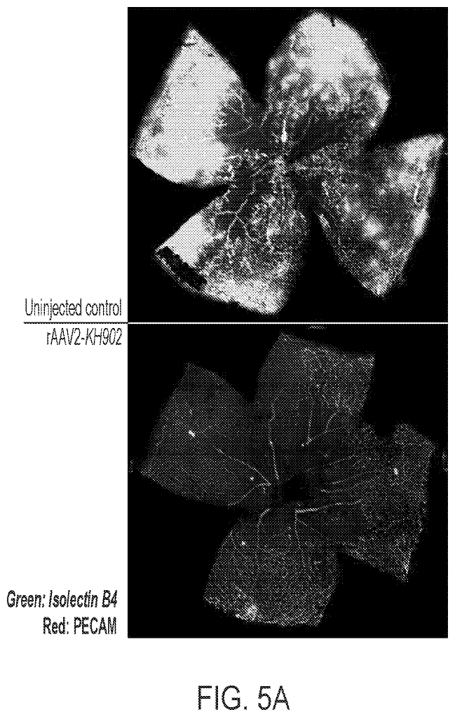

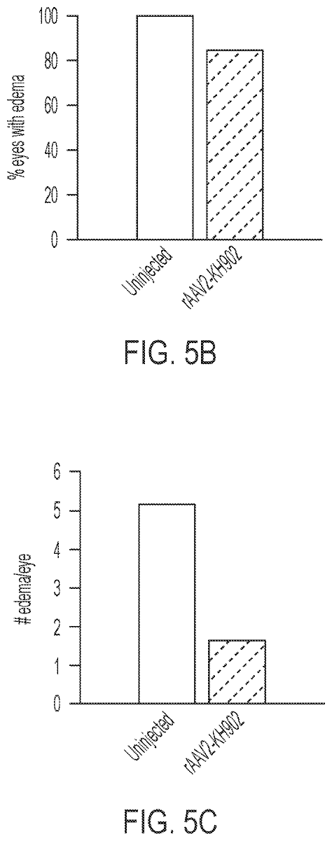

Injection of rAAV2-CBA-KH902 into the vitreous of neonatal mouse pups prevented normal retinal vascular development (FIG. 4). This data suggests that AAV-KH902 virions inhibited vascularization in vivo. To determine whether this vector design can inhibit choroidal neovascularization, the rAAV2-CBA-KH902 vector was tested in a mouse model for retinopathy of prematurity (ROP) (FIG. 5). Intravitreal injection of rAAV2-CBA-KH902 and subsequent hyperoxia treatment of mice led to a reduction in the percentage eyes with detectable edemas and the number of edemas in eyes of treated mice as compared to control uninjected eyes. This data suggests that AAV-KH902 is a potentially viable gene therapy platform for preventing and, possibly reversing, choroidal neovascularization.

### Example 2: Intravitreal Injection of AAV2 Vector is Effective at Delivering KH902 to Prevent Oxygen-Induced Retinopathy and Vascularization in Mice

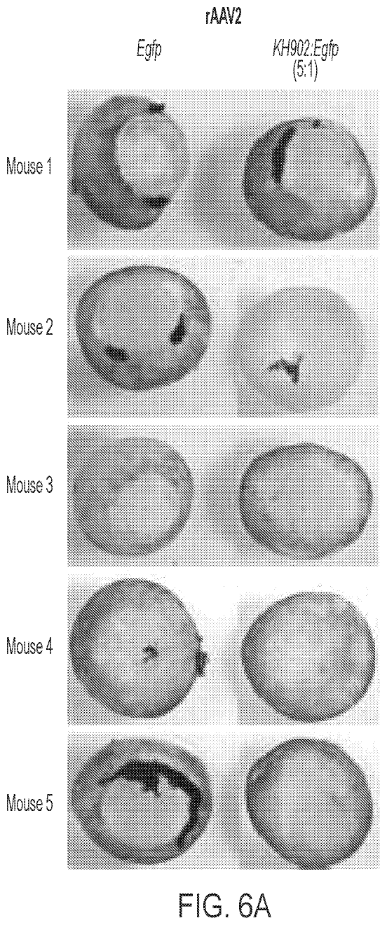

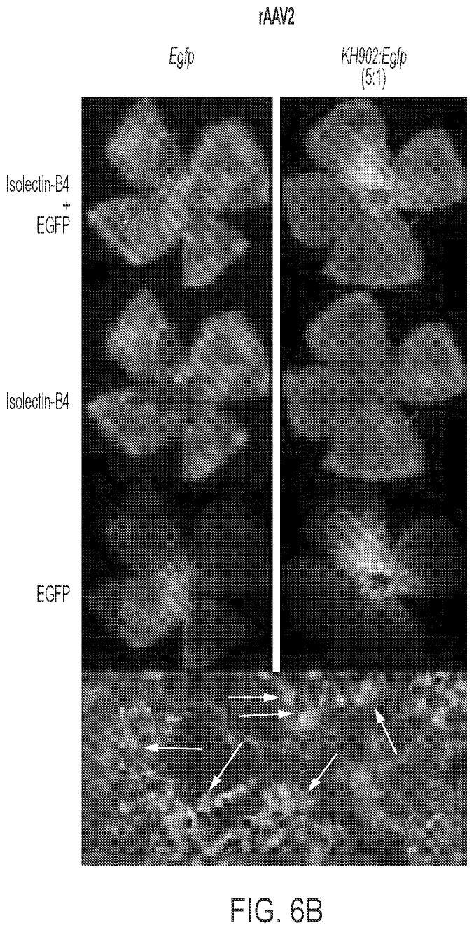

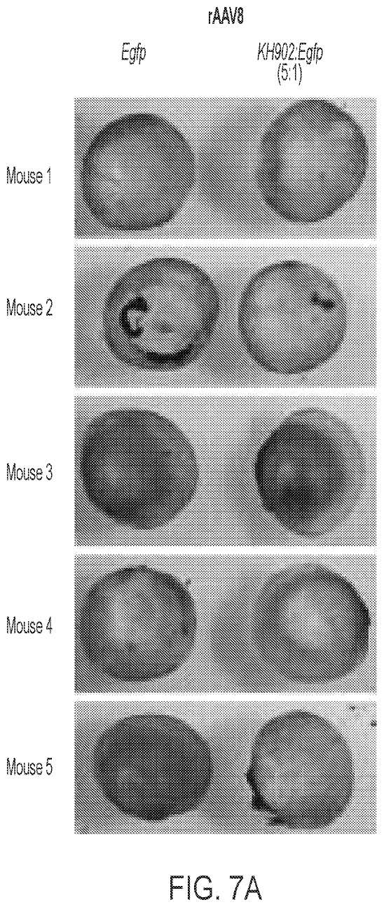

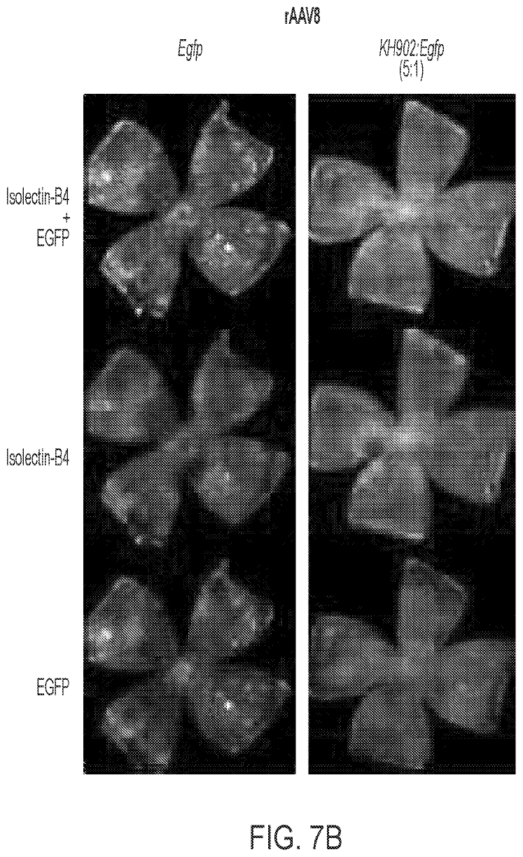

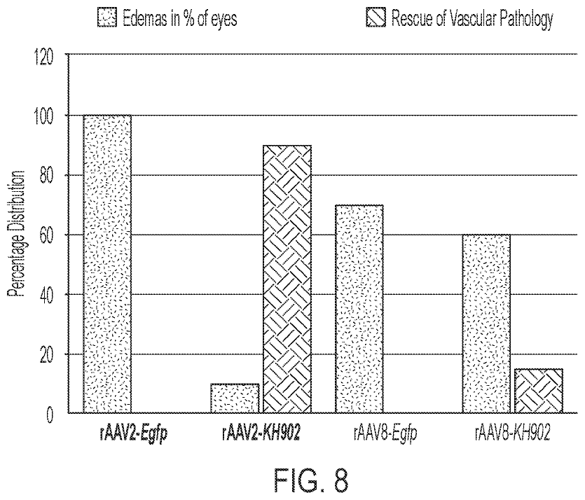

Neonatal mice were treated by intravitreal injection with vector at post-natal days (PN) 1-3. Each mouse was treated in one eye with vector packaging the EGFP transgene (rAAV-EGFP), and the opposing eye with a 5:1 ratio mixture of vector packaging the KH902 transgene (rAAV-KH902) and rAAV-EGFP, respectively. In all cases, the total dose was 1.5E⁹ vg per eye in a 1 μL volume. Mice were then kept at 70% oxygen until PN 7 and placed in normoxic conditions (20-21% oxygen) until PN 11. Mice were sacrificed at PN 18 and eyes were harvested and visualized (FIGS. 6A-6B and FIGS. 7A-7B). The pathology of treated eyes was then scored by visual inspection and scored (FIG. 8). Eyes treated with rAAV-EGFP alone are indicative for the extent of hyperoxia induction and serves as an internal control for variability of pathology. It should be noted that the absence of an edema does not mean that hyperoxia failed to induce retinopathy, nor does the presence of an edema in rAAV-KH902 treated eyes mean that the vector was non-effective. Rescue of vascular pathology is determined by the presence or absence of aneurysm nodules.

In rAAV2-EGFP treated eyes, which serve as negative controls, vascular pathologies were observed as a result of over proliferation and formation of vascular aneurysm nodules (FIG. 6B, bottom panel). Eyes treated with rAAV2-KH902 efficiently prevented the pathologies (FIGS. 6A-6B) and also reduced vascular development to a certain degree (FIG. 6B, right panels). In contrast, rAAV8-KH902 is very inefficient in preventing vascular pathologies (FIG. 7A-7B). This observation correlates with the low transduction of rAAV8-EGFP in retinal tissues (FIG. 7B, left panels). Nonetheless, in areas where there is some transduction, rAAV8-KH902 is able to partially prevent pathologies (FIGS. 7B, right panels and FIG. 8).

Overall, induction of retinopathies by hyperoxia worked in all mice even if the eye did not develop an edema (FIG. 8). Importantly, the KH902 transgene is able to reverse pathological vascularization with rAAV2, but poorly with rAAV8. However, the current results do not predict the outcome of treatments in adult animals, as regular vascular development is completed by then.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An isolated nucleic acid, comprising:
a transgene encoding an anti-vascular endothelial growth factor (anti-VEGF) agent, the
transgene being flanked by inverted terminal repeats (ITRs).

2. The isolated nucleic acid of claim 1, wherein the anti-VEGF agent is a human VEGF decoy receptor.

3. The isolated nucleic acid of claim 2, wherein the human VEGF decoy receptor comprises extracellular domain 2 of human VEGF receptor 1.

4. The isolated nucleic acid of claim 2, wherein the human VEGF decoy receptor comprises extracellular domains 3 and 4 of human VEGF receptor 2.

5. The isolated nucleic acid of any one of claims 2-4, wherein the VEGF decoy receptor is capable of binding to vascular endothelial growth factor (VEGF) and/or placenta growth factor (PlGF).

6. The isolated nucleic acid of claim 1 or 2, wherein the anti-VEGF agent is a human VEGF receptor fusion protein.

7. The isolated nucleic acid of claim 6, wherein the human VEGF receptor fusion protein comprises extracellular domain 2 of human VEGF receptor 1 fused to extracellular domains 3 and 4 of human VEGF receptor 2.

8. The isolated nucleic acid of claim 6, wherein the human VEGF receptor fusion protein comprises extracellular domain 2 of human VEGF receptor 1 fused to an Fc portion of an immunoglobulin.

9. The isolated nucleic acid of claim 6, wherein the human VEGF receptor fusion protein comprises extracellular domains 3 and 4 of human VEGF receptor 2 fused to an Fc portion of an immunoglobulin.

10. The isolated nucleic acid of claim 6, wherein the human VEGF receptor fusion protein comprises extracellular domain 2 of human VEGF receptor 1 fused to extracellular domains 3 and 4 of human VEGF receptor 2, and further fused to an Fe portion of an immunoglobulin. The isolated nucleic acid of claim 10, wherein the anti-VEGF agent is KH902.

12. The isolated nucleic acid of claim 10 or 11, wherein the an VEGF agent comprises an amino acid sequence at least 50%, at least 60%, at least 70%, at least 80% 90%, 99% or 100% identical to amino acid sequence of SEQ ID NO: 5, or a portion thereof.

13. The isolated nucleic acid of any one of claims 10-12, wherein the transgene comprises a nucleic acid sequence at least 50%, at least 60%, at least 70%, at least 80%, 90%, 99% or 100% identical to nucleic acid sequence of SEQ ID NO: 1 or a codon optimized variant thereof.

14. The isolated nucleic acid of any one of claims 6-13, wherein the anti-VEGF agent is capable of binding to anti-vascular endothelial growth factor (VEGF) and/or placenta growth factor (PlGF).

15. The isolated nucleic acid of any one of claims 1-14, wherein the isolated nucleic acid further comprises a promoter operably linked to the transgene.

16. The isolated nucleic acid of claim 15, wherein the promoter comprises cytomegalovirus (CMV) early enhancer.

17. The isolated nucleic acid of claim 16, wherein the promoter comprises a chimeric cytomegalovirus (CMV)/Chicken β-actin (CB) promoter.

18. The isolated nucleic acid of any one of claims 1 to 17, wherein the transgene comprises one or more introns.

19. The isolated nucleic acid of claim 18, wherein at least one intron is positioned between the promoter and the nucleic acid sequence encoding the anti-vascular endothelial growth factor (anti-VEGF) agent.

20. The isolated nucleic acid of any one of claims 1 to 19, wherein the transgene comprises a Kozak sequence. Io

21. The isolated nucleic acid of claim 20, wherein the Kozak sequence is positioned between the intron and the transgene encoding the anti-vascular endothelial growth factor anti-VEGF) agent.

22. The isolated nucleic acid of any one of claims 1 to 21, wherein the transgene comprises a 3′ untranslated region (3′UTR).

23. The isolated nucleic acid of any one of claims 1 to 22, wherein the transgene further comprises one or more miRNA binding sites.

24. The isolated nucleic acid of claim 23, wherein the one or more miRNA binding sites are positioned in a 3′UTR of the transgene.

25. The isolated nucleic acid of claim 23 or 24, wherein the at least one miRNA binding site is an immune cell-associated miRNA binding site.

26. The isolated nucleic acid of claim 25, wherein the immune cell-associated miRNA is selected from: miR-15a, miR-16-1, miR-17, miR-19a, miR-20a, miR-21, miR-29a/b/c, miR-30b, miR-31, miR-34a, miR-106a, miR-125a/b, miR-142-3p, miR-146a, miR-150, miR-155, miR-181a, miR-223 and miR-424, miR-221, miR-222, let-7i, miR-148, and miR-152.

27. The isolated nucleic acid of any one of claims 1-26, wherein the ITRs are adeno-associated virus ITRs of a serotype selected from the group consisting of AAV1 ITR, AAV2 ITR, AAV3 ITR, AAV4 ITR, AAV5 ITR, and AAV6 ITR.

28. The isolated nucleic acid of any one of claims 1-27, comprising a nucleic acid sequence at least 80%, 90% 99% or 100% identical to the nucleic acid sequence of SEQ ID NO: 2.

29. A vector comprising the isolated nucleic acid of any one of claims 1-28. to 30. The vector of claim 29, wherein the vector is a plasmid, a baculoviral vector, a rAAV vector, an Anelloviral vector or a ceDNA.

31. The vector of claim 29 or 30, wherein the vector comprises a nucleic acid sequence at least 60%, 70%, 80%, 90%, 95%, 99%, or 100% identical to the nucleic acid sequence of SEQ ID NO: 3.

32. A recombinant adeno-associated virus (rAAV) vector comprising a nucleic acid comprising, in 5′ to 3′ order:
(a) a 5′ AAV ITR;
(b) a CMV enhancer;
(c) a CBA promoter;
(d) a chicken beta-actin intron;
(e) a Kozak sequence;
(f) a transgene encoding an anti-VEGF agent, wherein the anti-VEGF agent is encoded by the nucleic acid sequence in SEQ ID NO: 1;
(g) a rabbit beta-globin polyA signal tail; and
(h) a 3′ AAV ITR.

33. A host cell comprising the isolated nucleic acid of any one of claims 1 to 28, or the vector of any one of claims 29-32.

34. The host cell of claim 33, wherein the host cell is a mammalian cell, yeast cell, bacterial cell, or insect cell.

35. A recombinant adeno-associated virus (rAAV) comprising:
an adeno-associated virus (AAV) capsid protein; and
the isolated nucleic acid of any one of claims 1-28.

36. A recombinant adeno-associated virus (rAAV) comprising:
an adeno-associated virus (AAV) capsid protein; and
the rAAV vector of claim 32.

37. The rAAV of claim 35 or 36, wherein the capsid protein is of a serotype selected from AAV1, AAV2, AAV3, AAV4, AAVS, AAV6, AAV7, AAVS, AAV9, and a variant of any of the foregoing.

38. The rAAV of any one of claim 35-37, wherein the capsid protein has tropism for ocular tissue.

39. The rAAV of claim 38, wherein the ocular tissue comprises ocular neurons, retina, sclera, choroid, retina, vitreous body, macula, fovea, optic disc, lens, pupil, iris, aqueous fluid, cornea, conjunctiva ciliary body, or optic nerve.

40. The rAAV of any one of claims 35-39, wherein the rAAV is a single-stranded AAV (ssAAV) or a self-complementary AAV (scAAV).

41. A pharmaceutical composition comprising the isolated nucleic acid of any one of claims 1-28, the vector of any one of claims 29-32, or the rAAV of any one of claims 35-40.

42. The pharmaceutical composition of claim 41, further comprises a pharmaceutically acceptable carrier.

43. The pharmaceutical composition of claim 41 or 42, wherein the pharmaceutical composition is formulated for intravitreal injection, intravenous injection, intratumoral injection, or intramuscular injection.

44. A method of inhibiting VEGF or PlGF activity in a subject in need thereof, the method comprising
administering to the subject a therapeutically effective amount of the isolated nucleic acid of any one of claims 1-28, the rAAV of any one of claims 35-40, or the pharmaceutical composition of any one of claims 41-43.

45. A method of delivering an anti-VEGF agent in a subject in need thereof, the method comprising
administering to the subject a therapeutically effective amount of the isolated nucleic acid of any one of claims 1-28, the rAAV of any one of claims 35-40, or the pharmaceutical composition of any one of claims 41-43.

46. A method of treating a neovascularization associated disease, an angiogenesis associated disease or a VEGF associated disease in a subject in need thereof, the method comprising
administering to the subject a therapeutically effective amount of the isolated nucleic acid of any one of claims 1-28, the rAAV of any one of claims 35-40, or the pharmaceutical composition of any one of claims 41-43.

47. The isolated nucleic acid of any one of claims 1-28, the rAAV of any one of claims 35-40, or the pharmaceutical composition of any one of claims 41-43 for use in inhibiting VEGF activity in a subject in need thereof.

48. The isolated nucleic acid of any one of claims 1-28, the rAAV of any one of claims 35-40, or the pharmaceutical composition of any one of claims 41-43 for use in delivering an anti-VEGF agent in a subject in need thereof.

49. The isolated nucleic acid of any one of claims 1-28, the rAAV of any one of claims 35-40, or the pharmaceutical composition of any one of claims 41-43 for use in treating a neovascularization associated disease, an angiogenesis associated disease, or a VEGF-associated disease in a subject in need thereof.

50. The method or the use of claims 44-49, wherein the delivery of the anti-VEGF agent results in at least 10%, at least 20%, at least 30%, at least 40%, at least 50%, at least 60%, at least 70%, at least 80%, at least 90%, or 100% inhibition of VEGF activity.

51. The method or the use of one of claims 44-50, wherein the subject s a non-human mammal.

52. The method or the use of claim 51, wherein the non-human mammal is mouse, rat, cat, dog, sheep, rabbit, horse, cow, goat, pig, guinea pig, hamster, chicken, turkey, or a non-human primate.

53. The method or the use of any one of claims 44-50, wherein the subject is a human.

54. The method or the use of claim 53, wherein the subject has or is suspected of having an angiogenesis associated disease or a VEGF associated disease.

55. The method or the use of claim 54, wherein the VEGF associated disease is a tumor, a cancer, a retinopathy, a wet age-related macular degeneration (wAMD), a macular edema, a choroidal neovascularization, or a corneal neovascularization.

56. The method or the use of any one of claims 44-55, wherein the administration comprises systemic administration, optionally wherein the administration is intravenous injection. The method or the use of any one of claims 44-56, wherein the administration comprises direct administration to ocular tissue, optionally wherein the direct administration is intravitreal injection, intraocular injection or topical administration.

58. The method or the use of any one of claims 44-57, wherein the administration results in delivery of the transgene to ocular tissue.

59. The method of claim 58, the ocular tissue comprises ocular neurons, retina, sclera, choroid, retina, vitreous body, macula, fovea, optic disc, lens, pupil, iris, aqueous fluid, cornea, conjunctiva ciliary body, or optic nerve.


SEQUENCE LISTING (10 sequences)

SEQ ID NO: 1 - DNA, 1659 bp, Artificial Sequence
  Synthetic
atggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - DNA, 4011 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 6832 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 4 - DNA, 6 bp, Artificial Sequence
  Synthetic
gccrcc

SEQ ID NO: 5 - protein, 552 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 6 - protein, 127 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 7 - protein, 195 aa, Artificial Sequence
  Synthetic
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPP

SEQ ID NO: 8 - protein, 322 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPP

SEQ ID NO: 9 - protein, 357 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 10 - protein, 425 aa, Artificial Sequence
  Synthetic
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK